(19) United States
(12) Patent Application Publication
(10) Pub. No.: US 2003/0165835 A1
(43) Pub. Date: Sep. 4, 2003

(54) Cell stress regulated human MHC class I gene

(75) Inventors: Thomas Spies, Seattle, WA (US); Veronika Spies, Seattle, WA (US)

Correspondence Address: Steven L. Highlander, Esq., FULBRIGHT & JAWORSKI L.L.P., Suite 2400, 600 Congress Avenue, Austin, TX, 78701, US

(73) Assignee: Fred Hutchinson Cancer Research Center Inc.

(21) Appl. No.: 09/855,612

(22) Filed: May 14, 2001

Related U.S. Application Data
(63) Continuation of application No. 09/303,161, filed on Apr. 29, 1999, now abandoned.
(60) Provisional application No. 60/029,044, filed on Oct. 29, 1996.

(30) Foreign Application Priority Data
Oct. 29, 1997 (WO) PCT/US97/20170

(51) Int. Cl. C12Q 1/68; G01N 33/574; A01K 67/027; A61K 48/00; A61K 39/395; C12N 5/08

(52) U.S. Cl. 435/6; 435/7.23; 435/366; 800/8; 800/18; 424/93.21; 424/155.1

(57) ABSTRACT

The present invention provides various methods by which the use of certain MHC-related molecules can be exploited in understanding and regulating the immune response. More particularly, the present invention describes the use of molecules expressed by certain cell types as markers, reagents and targets in the diagnosis and treatment of certain disease states including GVHD, cancer and the like.

### BACKGROUND OF THE INVENTION

The present application is a continuation-in-part of co-pending U.S. Provisional Patent Application Serial No. 60/029,044 filed Oct. 29, 1996. The entire text of the above-referenced disclosure is specifically incorporated by reference herein without disclaimer. The government may own rights in the present invention pursuant to grant numbers RO1 AI30581 and POI CA18221 from the National Institutes of Health.

1. Field of the Invention

The present invention relates generally to the field of immunology. More particularly, it concerns describes immune cell surface molecules that function as receptors for certain γ-δ T-cells which may be useful in the screening individuals for transplant suitability and the presence and treatment of cancer.

2. Description of Related Art

At its basis, the mammalian immune system is highly dependent on a wide variety of receptor-ligand interactions that often facilitate critical immune functions. Examples include the interactions between immune cells, such as the signalling between antigen-presenting cells and immune response cells, such as cytotoxic T-cells. Another example of an important receptor-ligand interaction is the binding of antibody to an invading pathogen, either causing its inactivation or signalling further receptor ligand interactions (macrophage, migration; complement cascade). Yet a further example of the highly receptor-ligand oriented nature of the human immune system is the ability, by virtue of a complex series of cell surface molecules, to distinguish self from non-self molecules (toxins; viral antigens) and cells (bacteria; tumor cells). The key to this self/non-self recognition system are the major histocompatibility molecules, or MHC.

The MHC are encoded in a region on chromosome six consisting of a large family of genes. These genes are responsible for the immune system's ability to distinguish between cells and substances that belong in the body and those that do not. MHC molecules bind peptides inside cells and transport them to the surface of every cell in the body, where they act as biological “ID” badges. White cells, including T-cells, which patrol the body to rid it of foreign invaders, ignore cells that display MHC molecules with bound peptides derived from the many proteins synthesized in normal cells. When a cell is infected by a virus or certain bacteria, peptides derived from degradation of proteins from these infectious agents are also presented by MHC molecules at the cell surface, thus marking the infected cell for destruction by T-cells. The MHC molecules, with the peptides they present, are the cell's passport because T-cells only recognize the antigen complex when it is associated with a foreign peptide.

The human MHC encodes the polymorphic class I and class II families of membrane-anchored glycoproteins that bind peptides in distinct subcellular compartments and present these on the cell surface to T-cells with αβ T-cell receptors. Class I molecules bind peptides derived from cytosolic protein degradation in the endoplasmic reticulum (ER) and thus enable cytotoxic T-cells to detect intracellular antigen, whereas class II molecules associate with peptides from extracellular sources in endosomal vesicles and activate helper T-cells. These functions are central to adaptive immune responses against microbial infections and depend on accessory molecules for antigen processing also encoded by genes in the MHC.

MHC alleles, specifically the HLA alleles, have been associated with certain diseases. Specific HLA antigens have a statistical association with certain presumed autoimmune disorders and lymphoid cell neoplasms. Among these disorders are a distinct set that are predominately associated with class I alleles and include sero-negative spondylarthropathies with the HLA-B27 gene, the association with Psoriasis vulgaris with HLA-Cw6 and Behcet disease with HLA-B51. Moreover, histocompatibility antigens are well known to be involved in graft rejection and graft versus host disease. Because of the role the MHC antigens play in graft rejection, histocompatibility typing of hosts and donors are carried out to minimize differences between donor and recipient.

One example where GVHD becomes prominent is bone marrow transplantation (BMT), which often is used in humans for the treatment of a variety of bone marrow related disorders and in cancer therapy to replace bone marrow cells lost to chemo- and radiotherapy. In genetically identical twins there are no immunological barriers to BMT, however in other circumstances genetic disparities result in immune-related complications including graft rejection and graft versus host disease.

BMT for the treatment of patients with a variety of malignancies and disorders is possible if a suitable donor may be located. Less than 30% of patients who might benefit from a transplant have identical siblings that could serve as donors, and only 3-5% have a donor with only one mismatched human lymphocyte antigen (HLA; a class of MHC molecules). Transplantation of unmodified bone marrow from the HLA-haploidentical two or three loci incompatible donors has been associated with unsuccessful outcome due to the high incidence of severe GVHD. Also the risk of graft failure may be 20% or higher. Extensive T-cell depletion of mismatched donor marrow can be used to prevent GVHD however, this has the undesirable consequence of an increased graft rejection in such transplants. In severe manifestations of GVHD the patients compromised immune system gives rise to complications in the gastrointestinal tract. These complications include diarrhea, anorexia, nausea and vomiting as well as malabsorption, abdominal pain ileus and ascites formation.

Thus, it is clear that a better understanding of immune recognition and the mechanisms responsible for diseases like GVHD is needed. Another potential area that requires further understanding is the ability of tumor cells to evade the recognition and destruction arm of the immune system.

### SUMMARY OF THE INVENTION

Therefore, it is a goal of the present invention to provide various methods by which the use of certain MHC-related molecules can be exploited in understanding and regulating the immune response. More particularly, it is a goal of the present invention to utilize molecules expressed by certain cell types as markers, reagents and targets in the diagnosis and treatment of certain disease states.

Thus in particular embodiments, there is provided a method for detecting a cancer cell in a sample comprising the steps of providing the sample; and identifying MICA or MICB expression in the sample. More particularly, the identifying comprises binding of MICA or MICB by a MICA- or MICB-binding agent. In preferred embodiments, the MICA- or MICB-binding agent is a first antibody. In yet more preferred embodiments, the first antibody is a bispecific antibody recognizing both MICA and MICB.

In certain aspects the first antibody may be labeled. The label may be a radiolabel, a fluorescent label, a chemilluminescent label, an enzyme, or a ligand. In other aspects, the first antibody is unlabeled and the first antibody is detected by binding of a detection agent to the first antibody. In particular embodiments, the detection agent is a second antibody. More particularly, the second antibody binds to an Fc-region of the first antibody, and in further embodiments, the second antibody may also be labeled. The binding of the first antibody may be a competitive binding with a second antibody.

In particular embodiments of this aspect of the present invention of MICA expression is identified, in other embodiments, MICB expression is identified, in still other aspects both MICA and MICB expression are identified. In other embodiments, the identifying comprises amplifying a MICA or MICB transcript. The amplifying may in particular embodiments, comprise PCR. In other embodiments, the amplifying may further comprise, prior to the PCR, reverse transcription. In other aspects of this method, the PCR product is detected by electrophoretic separation or following hybridization. In specific embodiments, the PCR is quantitative PCR.

In preferred embodiments of the present invention the sample is selected from the group consisting of lung tissue, skin tissue, muscle tissue, liver tissue, renal tissue, colon tissue, prostate tissue, breast tissue, brain tissue, cervical tissue, pancreatic tissue, stomach tissue, testicular tissue, ovarian tissue or marrow tissue. In other preferred embodiments, the sample is selected from the group consisting of sputum, blood, semen, plasma, serum, lymphatic fluid, urine and stool. In other aspects of the present invention, the cancer that is detected is selected from the group consisting of brain cancer, lymphatic cancer, liver cancer, stomach cancer, testicular cancer, cervical cancer, leukemia, melanoma, head & neck cancer, esophageal cancer, colon cancer, breast cancer, lung cancer, ovarian cancer, prostate cancer and renal cancer. In more specific embodiments, the cancer is colon cancer, breast cancer, lung cancer, ovarian cancer, prostate cancer or renal cancer.

The present invention also provides a method for purifying Vδ1 γδT cells comprising the steps of providing a MICA or MICB polypeptide fixed to a support; contacting the polypeptide with a starting cell population; and separating the support from the starting cell population to produce a purified Vδ1 γδT cells population. In certain embodiments, the method may further comprise washing the support following separation. In specific embodiments, the MICA polypeptide is fixed to the support. In other specific embodiments, the MICB polypeptide is fixed to the support. In still other embodiments, the MICA and MICB polypeptides are fixed to the support. The support independently may be a culture dish, a dipstick, a test tube, a column matrix, a bead, a filter membrane.

In more defined embodiments of the present invention, the starting cell population may independently comprise peripheral blood cells, lymph cells, or purified T-ells. The separating may comprise a centrifugation. In preferred embodiments, the purified Vδ1 γδT cell population comprises at least about 75% Vδ1 γδT cells, in other embodiments the purified Vδ1 γδT cell population may independently comprise at least about 80%, at least about 85%, at least about 90%, at least about 95%, at least about 97%, at least about 98%, at least about 99%/, at least about 99.9% or at least about 100% Vδ1 γδT cells.

Another aspect of the present invention provides a method for enriching a cell population for Vδ1 γδT cells comprising the steps of providing a MICA or MICB polypeptide linked to a fluorescent or chemilluminescent label; contacting the polypeptide with a starting cell population; and separating cells exhibiting fluorescence or chemilluminescence. In specific embodiments, the label is selected from the group consisting of fluorescein, rhodamine, green fluorescent protein and luciferase. More particularly, the separating comprises cell sorting. In particular embodiments, the polypeptide is linked directly to the label. In other embodiments, the polypeptide is linked to the label by a linking moiety. More particularly the linking moiety is a bead. In particular aspects the resulting cell population comprises at least about 99% Vδ1 γδT cells.

Also provided by the present invention is a method of targeting a therapeutic agent to a tumor cell expressing MICA or MICB on its surface comprising the steps of providing a therapeutic agent having a MICA- or MICB-binding agent conjugated thereto; and contacting the therapeutic agent with the tumor cell. In specific embodiments, the MICA- or MICB-binding agent is an antibody. In more specifically, the antibody may be a humanized murine monoclonal antibody. The therapeutic agent may be selected from the group consisting of a toxin, a cytokine, a nucleic acid encoding an antitumor agent, a chemotherapeutic and a radionuclide. The binding agent may bind to MICA, to MICB or alternatively the binding agent binds to both MICA and MICB.

The present invention further provides a method for treating cancer comprising the step of administering to a subject a therapeutic agent having a MICA- or MICB-binding agent conjugated thereto. In particular embodiments, the administering comprises injection. The injection may independently be intratumoral or intravenous.

Also contemplated by the present invention is a method for expanding Vδ1 γδT cells in a T-cell population comprising the steps of contacting the T-cell population with MICA or MICB; and incubating the T-cell population under conditions permitting the growth and division of T-cells. In particular embodiments the contacting comprises culturing the T cell population is cultured with cells expressing MICA and MICB. In other aspects, the contacting comprises providing MICA or MICB as purified proteins. The starting cell population may be selected from the group consisting of peripheral blood cells, lymph cells and purified T-cells.

In another aspect the present invention contemplates a method of adoptive immunotherapy comprising the step of administering to a patient a population of purified Vδ1 γδT cells. In specific embodiments, the patient suffers from cancer.

The present invention also provides independently, methods of increasing expression of MICA or MICB in a cell comprising providing to the cell an expression construct comprising a coding region for MICA or MICB, wherein the coding region is under the control of a promoter active in eukaryotic cells. More particularly, the expression construct may be a viral expression vector. In specific embodiments, the expression construct further comprises a polyadenylation signal. In more defined terms the cell is a human cell. The human cell may be located in a human subject. In other embodiments, the human cell is a tumor cell. In particular aspects the method may further comprise administering to the human subject a therapeutic agent having a MICA- or MICB-binding agent attached thereto.

Conversely, in alternate embodiments, the present invention contemplates that decreasing the expression of MICA and/or MICB may be useful in the treatment of disease, such as inflammatory bowel disease e.g. in Crohne's Disease.

Also contemplated by the present invention is a transgenic non-human mammal expressing a human MICA polypeptide. The mammal may be a mouse. In particular embodiments, the mouse expresses human MICA in a tissue specific fashion. In more specific embodiments, the mouse selectively expresses MICA in intestinal epithelium. Similarly, there is also provided by the present invention a transgenic non-human mammal expressing a human MICB polypeptide. In yet another alternative there is provided a transgenic non-human mammal expressing human MICA and MICB polypeptides.

These and other embodiments are described in greater detail in the following description and examples.

### BRIEF DESCRIPTION OF THE DRAWINGS

Not applicable

### DESCRIPTION OF ILLUSTRATIVE EMBODIMENTS

The present invention examines the role for the recently identified MHC-related cell surface molecules designated as MICA and MICB. The inventors have shown that these molecules are specifically expressed at the lining of the gastrointestinal (GI) tract. The GI tract is the primary site of infectious attack, is a major target for complications arising from GVHD and also plays a key role in the development of certain immune responses. Moreover, evidence is presented that suggests that these class I-like molecules are recognized by a subset of intestinal epithelial T-cells, having γδ-receptors, that presumably serve a specialized first-line immune defense against bacterial infection. Thus, the demonstration of MICA and MICB in this part of the anatomy indicates its likely involvement with one or more of these events.

The present inventors further have determined that MICA and MICB are expressed at the cell surface of colon and other cancer cells. In addition it is possible that these molecules may be exploited as diagnostic reagents for this or other types of tumors. Because of the unlikely role of MICA and MICB as ligands for T-cells they may have a role in anti-tumor immune response. Thus, any manipulations that result in the enhanced expression and/or the activity of the responsive T-cells may have a therapeutic value in cancer therapy.

The MICA and MICB genes also are intriguing from a gene regulation standpoint given the identification of heat shock response elements therein. Heat shock is a form of cellular stress which results in the induction of certain genes and their corresponding proteins to respond to the stress. This cellular machinery is, at least in part, the family of proteins known as heat shock proteins (HSPs). The inventors have shown that the MICA and MICB proteins of the present invention are regulated by a promoter heat shock elements similar to those of the HSP70 class of genes.

MICA Implications in Adoptive Immunotherapy and Graft Versus Host Disease

Adoptive immunotherapy is a therapeutic regimen involving the isolation and in vitro cloning and expansion of immunologically active cells from a donor. The expanded, therapeutically active cells are provided to a patient to obtain a therapeutic effect. If the donor is the patient, the transfer is “autologous.” If the donor is distinct from the patient, the transfer is “heterologous.” In heterologous transfers, the present invention provides for improved methods of matching donors with recipients. The principal behind adoptive immunotherapy is that immune effector cells, specifically are activated in vitro and administered to the patient to provide essential functions in mounting immune and anti-tumor responses.

It is proposed that, given the information linking MICA with the gut and its related immune functions, MICA may serve as an important marker for improving the results in transplant situations. More specifically, in addition to matching various HLA antigens, it may be equally (or more) important to look at MICA to determine the suitability of a particular donor-recipient combination. The screening methods applied would be the same as those currently employed for HLA typing.

One good example of how this could be exploited is the bone marrow transplant. BMT involves the transfer of hematopoeitic and immunocompetent lymphoid elements from a donor into a recipient. The major complication of bone marrow transplant has been the development of graft-versus-host disease. The clinical manifestations of GVHD produce a complex syndrome which reflects the graft versus host reaction as well as numerous infectious complications related to a compromised host immune system. In severe manifestations of GVHD, the patient's compromised immune system gives rise to complications in the gastrointestinal tract. These complications include diarrhea, anorexia, nausea and vomiting as well as malabsorption, abdominal pain, ileus and ascites formation. Clearly, a method that will alleviate such debilitating manifestations is needed. GVHD in bone marrow transplants is associated with alloreactivity of donor T lymphocytes against recipient cells. Alloreactivity requires several steps. The first is recognition of foreign tissue antigens.

The incidence of GVHD has been shown to be decreased in animals by performing a variety of typing studies to ensure the donor and host are suitably matched at the MHC locus. However, it has been shown that even when the donor and host are completely matched at the MHC locus, there remains a substantial GVHD in certain individuals. MICA and MICB may serve as targets for donor-derived infiltrating T-cells which may lead to an immune response against gut tissue of the marrow graft recipient. The present invention therefore provides a means of staging tissue damage in GVHD by using anti-MICA monoclonal antibodies and potentially provides a means to inhibit the role of MICA in eliciting this damaging pathology through the development of appropriate therapeutics.

Based on the knowledge that T-cells recognize antigens as small peptides in the context of major histocompatibility antigens, including human leukocyte antigens, several mechanisms of alloreaction have been proposed.

At the present time, it is believed that T-cells recognize transplanted cells as (1) intact foreign HLA molecule with an endogenous peptide that resembles a self HLA molecule with a previously seen foreign peptide, (2) a foreign HLA molecule-derived peptide in the context of a self HLA molecule, and (3) an intact empty foreign HLA molecule that again resembles a self HLA molecule with a foreign peptide. The mechanisms by which antigen is processed and presented within the context of MHC include (i) the presence of foreign HLA molecules on the surface of transplanted cells, (ii) the ingestion of foreign HLA molecules by self antigen-presenting cells (APC), (iii) the catabolism of these foreign HLA molecules, (iv) the presentation of peptides derived from these foreign HLA molecules in the context of self HLA molecules and (v) the processing and presentation of endogenously produced peptides in the context of self and foreign HLA molecules.

The processing of self or non-self protein takes place via different mechanisms. Exogenous proteins are endocytosed by APC and then transported to acid lysosomes where acid hydrolases cleave the protein into peptide antigens. These peptides are then loaded into HLA class II molecules that were previously occupied by invariant chains, and transported to the cell's surface. These exogenous peptides in the context of class II HLA molecules are recognized by CD4⁺lymphocytes. Endogenous peptides produced by antigen presenting cells are believed to associate with class I HLA molecules in the Golgi apparatus. These endogenous peptides in the context of class I HLA molecules are recognized by CD8⁺T lymphocytes. Alloreactivity also requires elaboration of costimulatory signals by the antigen-presenting cells, including the secretion of interleukin-1(IL-1), interleukin-6 (IL-6) and TNF-α. Finally, the T-cells themselves participate by secreting the T-cell growth factor, interleukin-2 (IL-2) and other is factors involved in T-cell regulation including interleukin-4 (IL4) and α2-interferon (α2-INF).

MICA is a polymorphic molecule that has been demonstrated herein to be an integral membrane, molecule present at the cell. As demonstrated herein for the first time, MICA expression is not associated with beta-2-microglobulin and is independent of cellular antigen processing. Furthermore, MICA and MICB are expressed almost exclusively in the gatrointestinal epithelium. MICA and MICB are unique among all class-I like molecules and their expression in gastrointestinal epithelia suggests the importance of MICA and MICB as transplantation antigens. Thus, one would identify the particular MICA and/or MICB allele present in a person to whom bone marrow cells would be provided. Then, the same determination would be made of potential donors. Matches then would further be screened for compatability on other bases, including HLA. The suitability of a donor will be determined based on the results of such screens.

MIC Implications In Cancer

The inventors have shown that MICA is expressed in various cancer cell lines suggesting that cells may be screened for the overexpression of MICA and/or MICB, its presence indicating potential carcinogenesis. The methods by which such screening takes place are well known in the art and, in many cases, described further below.

The present application describes an involvement of MICA with colon carcinoma, prostate, stomach, cervical, lymphatic, head and neck, esophagal, lung (small cell, NSC), liver (hepatocarcinoma), brain (glioma), breast, renal, melanoma, leukemia, testicular, ovarian or other cancer. The expression of MICA in tumor cells provides a marker for diagnostic screening methods for cancer in, for example, tumor or tissue biopsy samples. MICA and/or MICB expression in tumor cells may be detected and/or quantitated using for example, MICA or MICB binding agent such as a specific antibody or PCR primers suitable for amplification of mRNAs isolated from biopsy material. Thus the invention envisions diagnostic detection assays.

To the extent that MICA or MICB is expressed on tumor cells, it also is possible that one could target a therapy to these cells in vivo selectively. Such therapies may include the delivery of therapeutic agents including but not limited to toxins, cytokines, nucleic acids encoding anti-tumor agents, radio- or chemopharmaceuticals. Targeting may be effected by using MICA or MICB binding agents such as antibodies. Further, biological information on MICA and MICB and on the γδ T-cells may be used to enhance this interaction in patients thus helping T-cell anti-tumor immune response. In exploiting this previously unrecognized interaction, those of skill in the art will be able to rely on analogous strategies developed with other immunological systems to treat disease.

Heat Shock Proteins

The heat shock (HS) or stress response is a universal response occurring in organisms ranging from plants to primates. It is a response that can be elicited as a result of not only heat shock, but also as a result of a variety of other stresses including ischemia, anoxia, glucose deprivation, ionophores glucose and amino acid analogues, ethanol, transition series metals, drugs, hormones and bacterial and viral infections. This response is characterized by the synthesis of a family of well conserved proteins of varying molecular sizes that are differently induced and localized. These proteins are among the most phylogenetically conserved and are characterized according to their weights.

The transcriptional activation of stress protein-encoding genes occurs within minutes in response to environmental and or physiological trauma This speedy response has been attributed to the lack of introns in the vast majority of heat shock proteins. This absence of introns allows HSPs to circumvent a block in intron processing that occurs at elevated temperature. Thus, the HSP is translated with very high efficiency often at the expense of other proteins. As disclosed in more detail herein, MICA and MICB expression is induced by heat shock with kinetics that are similar to induction of HSP70 after heat shock. The identification of an operable heat shock promoter suggests that expression of MICA and MICB is coupled to cell stress. Thus these molecules may be recognized alone or complexed with ligands derived from heat shock proteins. The heat shock induction of MICA and MICB expression provides methods for detecting cell stress by detecting and/or measuring MICA or MICB expression as a cell stress marker. Within these embodiments, reagents are provided that are capable of expressing MICA or MICB and reagents that permit the detection of MICA or MICB expression on the surface of cells. These reagents may be used in methods and kit formulations for, for example, toxicity testing.

Given the inventors' observation regarding the heat shock regulatory elements found associated with MICA, it is possible that MICA may be connected to a heat shock or stress response. The heat shock-immune connection is not without precedent, as it has been shown that prokaryotic HSPs can elicit a strong immunological reaction, and some of the immunoglobulins and cytotoxic lymphocytes formed during an immune response are directed against peptides derived from HSPs. It appears that stress proteins which accumulate in response to environmental change may serve as prime targets in autoimmune disease. (Shinnick et al., 1988; Young, 1990; Rajaseker et al., 1990, Holoshitz et al., 1983). For example, rat adjuvant arthritis, which is a model for human rheumatoid arthritis in many respects, can be induced following injection with M. tuberculosis in complete Freund's adjuvant. It was shown that the arthritogenic agent in this composition was HSP65. Furthermore, T lymphocytes isolated from rheumatoid arthritis, tuberculosis and leprosy recognize mycobacterial stress proteins, and these cells seem to accumulate at sites of autoimmune lesions. These facts notwithstanding, it has been noted that not all T-cell clones that recognize these proteins are arthritogenic, and T lymphocytes that recognize stress proteins have been isolated from individuals who exhibit no signs of autoimmune disease. Thus, under normal circumstances, immunoregulatory mechanisms are involved in maintaining a balance between tolerance and autoimmunity with respect to self-reactive T-cells. The induction of heat shock may upset this balance and act as a trigger for immune defense.

There is a distinct advantage to having a pool of T-cells that is able to recognize well conserved protein determinants in that it provides the organism with a first line defense against common antigens. In accordance with this prediction, it has been demonstrated that in the mouse a portion of T-cells recognizing HSPs express the gamma delta T-cell receptor and that this population is preferentially enriched following heat shock and/or prior exposure to mycobacterial antigens. In any event, the integration of immune response and heat shock response further is evidenced by the dual characteristics of MICA immune function and heat shock regulation.

γδ-T-Cells

T lymphocytes most commonly express a T-cell receptor (TcR) consisting of a heterodimer of α- and β-chain polypeptides (Yanagi et al., 1984; Hedrick et al., 1984; Saito et al., 1984; Sim et al., 1984; Toyonaga and Mak, 1987). These cells recognize antigens in the context of self-molecules encoded by the major histocompatibility complex (MHC), and participate in regulating immune responses as well as killing target cells expressing foreign antigens. While the antigenic composition and biological role of αβ T-cells are well defined, the target(s) and function(s) of T-cells expressing γδ T-cell receptors remain obscure. For example, it has been difficult to identify ligands recognized by γδ receptors or to demonstrate classical MHC-restricted reactivity and furthermore, many of the previously obtained results are controversial.

Some types of ligands thus far identified for γδ T-cells are heat-shock proteins (HSP) and the purified protein derivative of mycobacterium (PPD), composed of a large fraction of HSP-65 (Augustin et al., 1989; Haregewoin et al., 1989; O'Brien et al., 1989; Holoshitz et al., 1989; Happ et al., 1989; Modlin et al., 1989; Janis et al., 1989; Kaufmann, 1990). HSPs are highly conserved among both prokaryotes and eukaryotes (Kaufmann, 1990). This implies that a significant proportion of γδ T-cells may be selected for reactivity to highly conserved antigens that can be derived both exogenously and endogenously. Recently phosphate compounds secreted by bacteria have been identified as non-peptide antigens recognized by many Vγ2Vδ2T cells.

In the past the critical unknown for γδ lymphocyte recognition of endogenous HSPs was whether and under what conditions these proteins or their peptide fragments would reach the cell surface in an immunogenic manner. During development, regulated expression of HSPs by immature stromal elements could be important in γδ recognition and function (Ferrick et al., 1990; Ferrick et al., 1989). Injured cells, induced by trauma to host tissue, may present stimulatory concentrations of self-antigens on their surfaces. The recognition of self-HSPs by γδ T-cells could then result in generating certain types of autoimmune disorders. Data consistent with such arguments have been made in the literature (Holoshitz et al., 1989; Modlin et al., 1989). The inventors observations suggest that Vδ1 γδ T-cells in the gut may interact with MICA and/or MICB or may bind to these molecules in complex with or without HSP-derived peptide fragments. Moreover, a linkage disequilibrium has been demonstrated between MICA alleles and closely linked HLA-B alleles suggesting that MICA may be involved in a number of autoimmume diseases such as Behcet disease. Thus, aberrant expression or mutant forms of MICA or MICB may be involved in the development of certain autoimmume diseases.

In a particular embodiment, the present invention provides a method for isolating or purifing γδ T cells from a population of starting cells by contacting a sample containing the Vδ1 γδT cells with MICA and/or MICB bound to a support and isolating the bound cells away from the remainder of the sample. The solid support may be any solid support known in the art such as a microtiter plate, a filter, culture dish, dipstick, column matrix, polystyrene beads, magnetic beads, agarose and the like. In those emboidments in which the solid support is a bead, the Vδ1 γδ T cells may be separated by centrifugation. The starting cell population may be any population of cells that comprises T-lymphocytes and more particularly γδ T cells, and even more preferably Vδ1 γδ T cells. Cells of the lymphoid system would be useful starting cells. The lymphoid system is composed of the primary lymphoid organs comprising the bone marrow and the thymus, the lymphocytes they produce and a collection of secondary lymphoid organs that are the sites where immune responses are initiated. The secondary lymphoid organs are interconnected by a circulatory system composed of two circulatory networks, the blood stream and the lymphatic. The lymphocytes are carried throughout most of the tissues and organs by this circulatory system. Thus, cells that are contemplated as the starting cell population herein include cells from the bone marrow, the thymus, the peripheral blood, as well as cells from the secondary organs such as the lymph nodes, the spleen, adenoids, Peyers patches and the appendix. The cell population may also comprise purified T-cells.

Another aspect of the present invention comprises a method for expanding Vδ1 γδT cells in a T-cell population. This method generally involves contacting the T-cell population with MICA or MICB; and incubating the T-cell population under conditions permitting the growth and division of T-cells. T-cell culture occurs under standard conditions familiar to those of skill in the art employing Lymphokines IL-2 and IL-7.

In specific examples, the present invention employs the rapid expansion method (REM) for quickly generating T cells. REM involves culturing the T-cells in association with a disproportionately large concentration of non-dividing feeder cells, present at in excess relative to the number of target T cells. Cultures grown under REM exhibit enhanced rates that can be elevated by the use of appropriate concentrations of additional feeder cells, IL2 and other growth factors. REM is described in detail in WO 96/06929 and 97/32970, which are incorporated herein by reference. As used herein feeder cells are accessory cell that provide co-stimulating functions in conjunction with T cell receptor activation. Thus in preferred embodiments, the feeder cells may be engineered to express MICA or MICB on their surface so as to stimulate the proliferation of Vδ1 γδT cells. In alternate embodiments, purified MICA or MICB peptides or variants thereof may be added to the culture medium in order to activate the Vδ1 γδT cells.

MICA, MICB and Related Polypeptides and Genes

MICA and MICB (MHC class I Chain-related genes A and B), are closely related and are encoded by 40 and 110 kilobases (kb) centromeric of HLA-B, respectively (Bahram et al., 1994). Distinct from all MHC class Ib genes, sequences directly homologous to MICA and MICB are conserved in many if not all mammals except rodents, and thus probably originated in primordial mammals or at an earlier evolutionary stage. The translation product of MICA is only distantly similar to mammalian MHC class I chains, but it shares the same domain organization and predictably a similar tertiary structure. An average of 25% of the MICA amino acids in the putative extracellular α1, α2, and α3 domains match residues in diverse human and mouse, or in any other mammalian MHC class I sequences (Bahram et al., 1994). A further characteristic of MICA is the complete absence of all of the residues implicated in the binding of CD8 and the presence of eight N-inked glycosylation sites in the α1-α3 domain sequences. Moreover, transcription of MICA is restricted to various epithelial cell lines and is not regulated by γ-interferon. MICB mRNA is present in the same cell lines, albeit at very low levels.

The inventors report the complete nucleotide sequence of the MICA gene comprising 11,722 basepairs (bp) of DNA 40 kilobases centromeric of HLA-B. The sequence was obtained from single-stranded (M13) and double-stranded (pUC19) templates of mapped or randomly shot-gun subcloned DNA fragments that were derived from the cosmid M32A (Spies et al., 1989). The first exon encoding the leader peptide is followed by an intron of 6840 bp, which is unusually large for a class I gene. The remainder of the MICA gene shows an organization quite similar to that of conventional class I genes, except for the presence of a relatively long intron following the transmembrane exon and the fusion of the cytoplasmic tail and 3′ untranslated sequence in a single last exon. The translated amino acid sequence corresponds to the MICA4 allele.

The MICB gene has been mapped in cloned cosmids by DNA blot hybridizations using a MICA cDNA probe. It corresponds to mRNA of about 2.4 kb, distinct from MICA mRNA, which is 1.4 kb in size (Bahram et al., 1994). A partial 2304 base pairs (bp) MICB cDNA clone lacking the leader peptide sequence was isolated from an IMR90 human lung fibroblast library by screening with the MICA cDNA probe. The missing 5′ end sequence was cloned by a 5′ Rapid Amplification of cDNA ends polymerase chain reaction (RACE-PCR) procedure (5′-AMPLIFINDER RACE kit; Clontech, Palo Alto, Calif.) after reverse transcription (RT) of poly(A)⁺HeLa cell mRNA in the presence of a specific RT primer (3′-ACTGGGGAACAAGGTTTATATGAGA-5′, MICB nucleotides 1653-1677; SEQ ID NO:5). Purified first-strand cDNA was ligated to a 5′ anchor oligonucleotide with T4 RNA polymerase, and amplified by PCR using anchor primer and an MICB oligonucleotide (3′-TGTCACCCGTCTTCTACAGGACCC-5′, MICB nucleotides 215-238; SEQ ID NO:6). The amplified 250 bp DNA fragment was directly cloned in PCRII (Invitrogen, San Diego, Calif.) and sequenced. A cDNA including the complete MICB coding sequence was subsequently generated by RT-PCR and cloned, using the same RT primer and PCR primers flanking the single long open reading frame [5′-(Sal I)GGGGCCATGGGGCTGGG-3′ SEQ ID NO:7, and 3′-ATCTGAGATGTCGGTCC-(Bam HI)-5′ SEQ ID NO:8).

The full-length MICB cDNA sequence of 2376 bp encodes a polypeptide of 383 amino acids that begins with a probable translation initiation codon (ATG) at nucleotide position 6 (Kozak, 1991) and has a predicted Mᵣ of 43000. The stop codon is followed by a relatively long 3′ untranslated region, which accounts for the size difference of the MICB and MICA mRNAs. A consensus polyadenylation signal near the 3′ end of the MICB cDNA is missing; the nearest AATAAA sequence is located 772 bp upstream and an appropriately positioned alternative polyadenylation signal is not readily discernible (Wickens, 1990).

The MICB translation product is identical to the MICA chain in length and domain organization and is highly similar, with 83% matching amino acid residues. Of the total of 65 amino acid substitutions, 18 are clustered within a segment of 24 amino acids in the putative transmembrane segment of MICB, which represents the sole highly disparate portion of the aligned sequences. This difference could affect the intracellular trafficking of the MICB chain or its assembly with other protein subunits. In the α1-α3 domains, MICB and MICA share 86% amino acid sequence similarity, with 15, 14, and 8 amino acid substitutions in the α1, α2, and α3 domains, respectively, which show no notable preferential distribution. Like MICA, the putative MICB chain may be heavily glycosylated, owing to the presence of five potential N-linked glycosylation sites, of which four in the α3 domain are common to both sequences. None of the three N-inked glycosylation motifs in MICA α1 and α2 are conserved in MICB, which has one such motif in the α2 domain. The highly conserved glycosylation site at amino acid position 86 in MHC class I chains is missing in MICB and MICA. Both sequences include the two pairs of cysteines in the α2 and α3 domains, which form intradomain disulfide bonds in class I chains, and several extra cysteine residues.

Common to MICB and MICA is a gap in the α1 domain, which corresponds to the peptide side chain-binding pocket B (“45” pocket) in many MHC class I chains, and an insertion of 6 amino acids at position 147 in the α2 domain (Bahram et al., 1994). These alterations could result in occlusion of the putative peptide-binding site, possibly with the same effect as in the mouse T10^{b} and T22^{b} class Ib claims, which have gaps of 3 and 13 amino acids at the positions 47 and 142 in the α1 and α2 domains, respectively, and apparently do not associate with peptides (Schild et al., 1994; Kaliyaperumal et al., 1995). Overall, MICB shows the same degree of divergence from mammalian MHC class I chains as MICA, with most of the amino acid residues that are invariant among vertebrate class I sequences being conserved (Grossberger and Parham, 1992; Bahram et al., 1994). Thus, altogether, MICB and MICA are very closely related and were probably derived by a relatively recent gene duplication.

Additional sequences similar to MICA and MICB (MICC, MICD, and MICE) have been localized in the human MHC near the HLA-E, -A, and -F genes using yeast artificial chromosome (YAC) clones spanning the class I region (Bahram et al., 1994). By partial genomic sequencing of corresponding cosmid DNA, these three sequences were identified as truncated gene fragments. Thus, MICA and MICB are the only functional members in this family of highly diverged MHC class I genes. This is similar to the existence of numerous class I pseudogenes and gene fragments in the human MHC and mouse H2 complex (Stroynowski, 1990; Geraghty, 1993).

The MICB gene, like MICA, encodes mRNA directing the synthesis of a functional polypeptide. These genes represent the most divergent mammalian MHC class I genes known and share a common origin that probably predates the main mammalian radiation. Their evolutionary preservation, indicates selection for an adaptive function. This is supported by the findings reported herein below that both genes are regulated by promoter heat-shock response elements related to those of HSP70 genes, and by results indicating the cell surface expression of MICA protein. In addition, MICA is highly polymorphic with sixteen allelic variants so far identified. This degree of sequence variation is characteristic of the well known MHC class I and class II proteins. A probable immunological function of MICA and MICB is demonstrated by the findings disclosed hereinbelow.

The MICA, MICB genes or their corresponding cDNA can be inserted into an appropriate cloning vehicle for manipulation thereof. In addition, sequence variants of the polypeptide can be prepared. These may, for instance, be minor sequence variants of the polypeptide that arise due to natural variation within the population or they may be homologues found in other species. They also may be sequences that do not occur naturally but that are sufficiently similar that they function similarly and/or elicit an immune response that cross-reacts with natural forms of the polypeptide. Sequence variants can be prepared by standard methods of site-directed mutagenesis such as those described below in the following section.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide are identified by an empirical approach in which portions of the gene encoding the polypeptide are expressed in a recombinant host, and the resulting proteins tested for their ability to elicit an immune response. For example, PCR can be used to prepare a range of cDNAs encoding peptides lacking successively longer fragments of the C-terminus of the protein. The immunoprotective activity of each of these peptides then identifies those fragments or domains of the polypeptide that are essential for this activity. Further experiments in which only a small number of amino acids are removed at each iteration then allows the location of the antigenic determinants of the polypeptide.

Another embodiment for the preparation of the polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

Within one embodiment of the invention, soluble forms of MICA or MICB are produced by recombinant expression of a truncated MICA or MICB coding regions. Within a preferred embodiment, a truncated MICA or MICB lacks the transmembrane domain and cytoplasmic tail and includes the three extracellular domains. Such truncated forms of the invention may be expressed from suitable host cells including yeast, mammalian, and insect cells using regulatory sequences, vectors and methods well established in the literature. To facilitate purification and/or identification of the truncated molecules, it may be preferable to include a sequence encoding a tag. The use of antigenic and other tags are well established and include Myc-tags, hemaglutinin tags and His tags. His tags in which the cloning sequence of interest is joined in-frame with a sequence encoding oligomeric histidines permit the purification of the resulting proteins using metal-affinity chromatography. Soluble MICA or MICB proteins produced in this manner may be used to block the function of MICA or MICB by competing with proteins that interact with MICA or MICB. Such soluble molecules may have value not only in functional studies, but may also be useful in blocking T-cell recognition of MICA or MICB. The soluble molecules may also be exploited to derive minimal peptides or other agents that have powerful effects in blocking T-cell function. Further, soluble peptides may also be useful in adoptive immunotherapy.

Successful applications of the peptide mimetic concept have thus far focused on mimetics of β-turns within proteins, which are known to be highly antigenic. Likely β-turn structure within a polypeptide can be predicted by computer-based algorithms as discussed above. Once the component amino acids of the turn are determined, peptide mimetics can be constructed to achieve a similar spatial orientation of the essential elements of the amino acid side chains.

Modification and changes may be made in the structure of a gene and still obtain a functional molecule that encodes a protein or polypeptide with desirable characteristics. The following is a discussion based upon changing the amino acids of a protein to create an equivalent, or even an improved, second-generation molecule. The amino acid changes may be achieved by change the codons of the DNA sequence, according to the following data.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982).

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0+1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Site-Specific Mutagenesis

Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 nucleotides on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

Antisense

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-standed (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Genetic Constructs and Their Delivery to Cells

I. Genetic Constructs

Within certain embodiments expression vectors are employed to express various genes to produce large amounts of the MICA or MICB polypeptide product, which can then be purified and, for example, be used to vaccinate animals to generate antisera or monoclonal antibody with which further studies may be conducted. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the MICA and MICB products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter refers to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Table 2 lists several inducible elements/promoters which may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct. Enhancer/promoter elements contemplated for use with the present invention include but are not limited to Immunoglobulin Heavy Chain, Immunoglobulin Light, Chain T-Cell Receptor, HLA DQ α and DQ β, β-Interferon, Interleukin-2, Interleukin-2 Receptor, MHC Class II 5, MHC Class II HLA-DRα, β-Actin, Muscle Creatine Kinase, Prealbumin (Transthyretin), Elastase I, Metallothionein, Collagenase, Albumin Gene, α-Fetoprotein, τ-Globin, β-Globin, e-fos, c-HA-ras, Insulin, Neural Cell Adhesion Molecule (NCAM), α1-Antitrypsin, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular-Dystrophy, SV40, Polyoma, Retroviruses, Papilloma Virus, Hepatitis B Virus, Human Immunodeficiency Virus, Cytomegalovirus, Gibbon Ape Leukemia Virus. Inducible promoter elements and their associated inducers are listed in Table 2 below.

TABLE 2

| Element | Inducer |
|---|---|
| MT II | Phorbol Ester (TPA), Heavy metals |
| MMTV (mouse mammary | Glucocorticoids |
| tumor virus) | |
| β-Interferon | poly(rI)X, poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating | Thyroid Hormone |
| Hormone α Gene | |
| Insulin E Box | Glucose |

In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). Adeno-associated viruses are also useful in this context (Ridgeway, 1988; Baichwal and Sugden, 1986; Hermonat and Muzycska, 1984). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

II. Delivery of Genetic Constructs

There are a number of ways in which nucleic acids may introduced into cells. Several methods, including viral and non-viral transduction methods, are outlined below.

(i) Adenovirus

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement-has been detected after extensive amplification. Adenovirus can infect virtually all epithelial cells regardless of their cell cycle stage. So far, adenoviral infection appears to be linked only to mild disease such as acute respiratory disease in humans.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by AdS DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra Kb of DNA. Combined with the approximately 5.5 Kb of DNA that is replaceable in the E1 and E3 regions, the maximum capacity of the current adenovirus vector is under 7.5 Kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grnmhaus and Horwitz, 1992; Graham and Prevec, 1992). Recently, animal studies suggested that recombinant adenovirus could be used for gene therapy (Stratford-Pernicaudet and Perricaudet, 1991; Stratford-Perricaudet et a., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

(ii) Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calciun phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

(iii) Adeno-Associated Virus (AAV)

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al. 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

(iv) Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) and herpes viruses may be employed. They offer several attractive features for various mammalian cells (Friedmann, 1989; Ridgeway, 1988; Baichwal and Sugden, 1986; Coupar et al., 1988; Horwich et al., 1990).

In vitro studies of hepatitis B viruses showed the virus retained the ability for helper-dependent packaging and reverse transcription despite the deletion of up to 80% of its genome (Horwich et al., 1990), suggesting that large portions of the genome could be replaced with foreign genetic material. The hepatotropism and persistence (integration) are particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

(v) Non-Viral Vectors

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al., (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring naked DNA expression constructs into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin,- and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CDS (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virally any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent T-cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to diameter ratio is 10:1. The airlift reactor scales up relatively easily, has good mass transfer of gases and generates relatively low shear forces.

Transgenic Animals

In one embodiment of the invention, transgenic animals are produced which contain a functional transgene encoding a functional MICA or MICB polypeptide or variants thereof. Transgenic animals expressing MICA or MICB transgenes, recombinant cell lines derived from such animals and transgenic embryos may be useful in methods for screening for and identifying agents that induce or repress expression of the transgene or which interfere with the function performed by MICA or MICB. Agents that cause cell stress will cause the expression of the MICA or MICB transgene. Thus transgenic animals, embryos and cell lines of the invention can also be used in toxicity screens as indicators of cell stress. Transgenic animals of the invention can also be used as models for studying indications such as graft-versus host disease and colon cancers.

In one embodiment of the invention, a human MICA or MICB transgene is introduced into a non-human host to produce a transgenic animal expressing a human MICA or MICB gene. Within a preferred embodiment, the non-human host does not normally contain a MICA or MICB gene. The transgenic animal is produced by the integration of the human MICA or MICB transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985 (which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety). It may be desirable to replace the endogenous MICA or MICB by homologous recombination between the transgene and the endogenous gene or the endogenous gene may be eliminated by deletion as in the preparation of “knock-out” animals. Typically, a MICA or MICB gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a MICA or MICB transgene. The absence of MICA or MICB genes in mice permit the study of MICA or MICB expression in an environment free of competing endogenous MICA or MICB sequences.

As noted above, transgenic animals and cell lines derived from such animals may find use in toxicity testing. In this regard, transgenic animals and cell lines capable of expressing human MICA or MICB may be exposed to test substances under conditions in which such animals and/or cell lines, in the absence of the test substance, do not express human MICA or MICB. The animals and/or cell lines are then tested for the expression of MICA or MICB. Animals may be tested for MICA or MICB expression by biopsy of tissues using MICA or MICB specific antibodies or using PCR assays designed to detect MICA or MICB mRNA production. In a similar manner cell lines exposed to test substances are also assayed for MICA or MICB expression. Within these methods test assays are compared to MICA or MICB expression in control animals or cell lines that have not been exposed to the test substance. The detection of MICA or MICB expression following exposure to a test substance indicates that cells undergo physiological stress in the presence of such a substance.

Purification of MICA, MICB and Related Polypeptides

In certain embodiments of the invention, it will be desirable to produce functional MICA or MICB polypeptide or variants thereof. Protein purification techniques are well known to those of skill in the art. These techniques tend to involve the fractionation of the cellular milieu to separate the MICA, MICB or related polypeptides from other components of the mixture. Having separated MICA, MICB and related polypeptides from the other plasma components the MICA, MICB or related polypeptide sample may be purified using chromatographic and electrophoretic techniques to achieve complete purification. Analytical methods particularly suited to the preparation of a pure peptide are ion-exchange chromatography, exclusion chromatography; polyacrylamide gel electrophoresis; isolectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state, ie., in this case, relative to its purity within a hepatocyte or β-cell extract. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “−fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

Synthetic Polypeptides

The present invention also describes MICA, MICB and related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

Detection And Quantitation Of Nucleic Acid Species

One embodiment of the instant invention comprises a method for identification of MICA or MICB expression in a biological sample by amplifying and detecting nucleic acids corresponding to MICA or MICB mutants. The biological sample can be any tissue or fluid in which these mutants might be present. Various embodiments include cells from the lung, muscle, liver, renal, prostate, breast, cervical, pancreatic, stomach, testicular, ovarian, gastrointestinal tract, the thymus, bone marrow aspirate, bone marrow biopsy, lymph node aspirate, lymph node biopsy, spleen tissue, fine needle aspirate, skin biopsy or organ tissue biopsy. Other embodiments include samples where the body fluid is peripheral blood, serum, plasm, semen, lymph fluid, ascites, serous fluid, pleural effusion, sputum, cerebrospinal fluid, lacrimal fluid, stool or urine.

Nucleic acid used as a template for amplification is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to MICA or MICB and variants thereof are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994).

Following detection, one may compare the results seen in a given patient with a statistically significant reference group of normal patients and patients that have for example, cancer, inflammatory bowel disease or a defect involving MICA or MICB. In this way, it is possible to correlate the amount of MICA, MICB or variants thereof detected with various clinical states.

1. Primers

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

2. Template Dependent Amplification Methods

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thiol-triphosphates in one strand of a restriction site may also be useful in the amplification of nucleic acids in the present invention, Walker et al., (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88110315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as 17 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by 17 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: PCR PROTOCOLS: A GUIDE TO METHODS AND APPLICATIONS, Academic Press, N.Y., 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

3. RNase Protection Assay

Methods for screening by identifying mutations associated with diseases such as GVHD must be able to assess large regions of the genome. Once a relevant mutation has been identified in a given patient, other family members and affected individuals can be screened using methods which are targeted to that site. The ability to detect dispersed point mutations is critical for genetic counseling, diagnosis, and early clinical intervention as well as for research into the etiology of cancer and other genetic disorders. The ideal method for genetic screening would quickly, inexpensively, and accurately detect all types of widely dispersed mutations in genomic DNA, cDNA, and RNA samples, depending on the specific situation.

Historically, a number of different methods have been used to detect point mutations, including denaturing gradient gel electrophoresis (“DGGE”), restriction enzyme polymorphism analysis, chemical and enzymatic cleavage methods, and others (Cotton, 1989). The more common procedures currently in use include direct sequencing of target regions amplified by PCR™ and single-strand conformation polymorphism analysis (“SSCP”).

Another method of screening for point mutations is based on RNase cleavage of base pair mismatches in RNA/DNA and RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single and multiple base point mutations. U.S. Pat. No. 4,946,773 describes an RNase A mismatch cleavage assay that involves annealing single-stranded DNA or RNA test samples to an RNA probe, and subsequent treatment of the nucleic acid duplexes with RNase A. After the RNase cleavage reaction, the RNase is inactivated by proteolytic digestion and organic extraction, and the cleavage products are denatured by heating and analyzed by electrophoresis on denaturing polyacrylamide gels. For the detection of mismatches, the single-stranded products of the RNase A treatment, electrophoretically separated according to size, are compared to similarly treated control duplexes. Samples containing smaller fragments (cleavage products) not seen in the control duplex are scored as “+”.

Currently available RNase mismatch cleavage assays, including those performed according to U.S. Pat. No. 4,946,773, require the use of radiolabeled RNA probes. Myers and Maniatis in U.S. Pat. No. 4,946,773 describe the detection of base pair mismatches using RNase A Other investigators have described the use of E. coli enzyme, RNase I, in mismatch assays. Because it has broader cleavage specificity than RNase A, RNase I would be a desirable enzyme to employ in the detection of base pair mismatches if components can be found to decrease the extent of non-specific cleavage and increase the frequency of cleavage of mismatches. The use of RNase I for mismatch detection is described in literature from Promega Biotech. Promega markets a kit containing RNase I that is shown in their literature to cleave three out of four known mismatches, provided the enzyme level is sufficiently high.

The RNase protection assay as first described by Melton et al (1984) was used to detect and map the ends of specific mRNA targets in solution. The assay relies on being able to easily generate high specific activity radiolabeled RNA probes complementary to the mRNA of interest by in vitro transcription. Originally, the templates for in vitro transcription were recombinant plasmids containing bacteriophage promoters. The probes are mixed with total cellular RNA samples to permit hybridization to their complementary targets, then the mixture is treated with RNase to degrade excess unhybridized probe. Also, as originally intended, the RNase used is specific for single-stranded RNA, so that hybridized double-stranded probe is protected from degradation. After inactivation and removal of the RNase, the protected probe (which is proportional in amount to the amount of target mRNA that was present) is recovered and analyzed on a polyacrylamide gel.

The RNase Protection assay was adapted for detection of single base mutations by Myers and Maniatis (1985) and by Winter and Perucho (1985). In this type of RNase A mismatch cleavage assay, radiolabeled RNA probes transcribed in vitro from wild type sequences, are hybridized to complementary target regions derived from test samples. The test target generally comprises DNA (either genomic DNA or DNA amplified by cloning in plasmids or by PCR™), although RNA targets (endogenous mRNA) have occasionally been used (Gibbs and Caskey, 1987; Winter and Perucho, 1985). If single nucleotide (or greater) sequence differences occur between the hybridized probe and target, the resulting disruption in Watson-Crick hydrogen bonding at that position (“mismatch”) can be recognized and cleaved in some cases by single-strand specific ribonuclease. To date, RNase A has been used almost exclusively for cleavage of single-base mismatches, although RNase I has recently been shown as useful also for mismatch cleavage. There are recent descriptions of using the MutS protein and other DNA-repair enzymes for detection of single-base mismatches (Ellis et al., 1994; Lishanski et al., 1994).

By hybridizing each strand of the wild type probe in RNase cleavage mismatch assays separately to the complementary Sense and Antisense strands of the test target, two different complementary mismatches (for example, A-C and G-U or G-T) and therefore two chances for detecting each mutation by separate cleavage events, was provided. Myers et al. (1985) used the RNase A cleavage assay to screen 615 bp regions of the human β globin gene contained in recombinant plasmid targets. By probing with both strands, they were able to detect most, but not all, of the β-globin mutations in their model system. The collection of mutants included examples of all the 12 possible types of mismatches between RNA and DNA: rA/dA, rC/dC, rU/dC, rC/dA, rC/dT, rU/dG, rG/dA, rG/dG, rU/dG, rA/dC, rG/dT, and rA/dG.

Myers et. al. (1985) showed that certain types of mismatch were more frequently and more completely cleaved by RNase A than others. For example, the rC/dA, rC/dC, and rC/dT mismatches were cleaved in all cases, while the rG/dA mismatch was only cleaved in 13% of the cases tested and the rG/dT mismatch was almost completely resistant to cleavage. In general, the complement of a difficult-to-detect mismatch was much easier to detect. For example, the refractory rG/dT mismatch generated by probing a G to A mutant target with a wild type sense-strand probe, is complemented by the easily cleaved rC/dA mismatch generated by probing the mutant target with the wild type antisense strand. By probing both target strands, Myers and Maniatis (1986) estimated that at least 50% of all single-base mutations would be detected by the RNase A cleavage assay. These authors stated that approximately one-third of all possible types of single-base substitutions would be detected by using a single probe for just one strand of the target DNA (Myers et al., 1985).

In the typical RNase cleavage assays, the separating gels are run under denaturing conditions for analysis of the cleavage products. This requires the RNase to be inactivated by treating the reaction with protease (usually Proteinase K, often in the presence of SDS) to degrade the RNase. This reaction is generally followed by an organic extraction with a phenol/chloroform solution to remove proteins and residual RNase activity. The organic extraction is then followed by concentration and recovery of the cleavage products by alcohol precipitation (Myers et al., 1985; Winter et al., 1985; Theophilus et al., 1989).

4. Separation Methods.

Following amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

5. Identification Methods

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify allelic variants of MICA or MICB using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. As disclosed herein, a human MICA or MICB gene and cDNA have been cloned. Using these reagents, oligonucleotide primers may be designed to permit the amplification of sequences in the MICA or MICB gene that may then be analyzed by either direct sequencing to identify specific allele sequences in the MICA or MICB gene. Particularly preferred regions for designing oligonucleotide primers include intron-exon junctions, preferably from flanking intron sequences. Methods for DNA sequence based typing have been disclosed for example by Santamaria et al. (WO 92/19771 which is incorporated herein by reference in its entirety) and Tilanus (WO 92/08117; which is incorporated herein by reference in its entirety).

6. Kit Components

All the essential materials and reagents required for detecting MICA, MICB, or alleles and variants thereof, in a biological sample may be assembled together in a kit. This generally will comprise preselected primers for specific markers. Also included may be enzymes suitable for amplifying nucleic acids including various polymerases (RT, Taq, etc.), deoxynucleotides and buffers to provide the necessary reaction mixture for amplification.

Such kits generally will comprise, in suitable means, distinct containers for each individual reagent and enzyme as well as for each marker primer pair. Preferred pairs of primers for amplifying nucleic acids are selected to amplify the sequences specified in SEQ ID NO:1, SEQ ID NO:3, the cDNAs for MICA and MICB.

In another embodiment, such kits will comprise hybridization probes specific for MICA, MICB or any variant thereof, chosen from a group including nucleic acids corresponding to the sequences specified in SEQ ID NO1, SEQ ID NO:3, or any variant thereof. Such kits generally will comprise, in suitable means, distinct containers for each individual reagent and enzyme as well as for each marker hybridization probe.

7. RNA Fingerprinting

RNA fingerprinting technology has been demonstrated as being effective in identifying genes that are differentially expressed in cancer (Liang et al., 1992; Wong et al., 1993; Sager et al., 1993; Mok et al., 1994; Watson et al., 1994; Chen et al., 1995; An et al., 1995). The present invention utilizes the RNA fingerprinting technique to identify genes that may be differentially expressed in various diseased states such as GVHD, colon cancers and the like.

RNA fingerprinting is a means by which RNAs isolated from many different tissues, cell types or treatment groups can be sampled simultaneously to identify RNAs whose relative abundances vary. Two forms of this technology were developed simultaneously and reported in 1992 as RNA fingerprinting by differential display (Liang and Pardee, 1992; Welsh et al., 1992). (See also Liang and Pardee, U.S. Pat. No. 5,262,311, incorporated herein by reference in its entirety.) Some of the experiments described herein were performed similarly to Donahue et al., 1994.

All forms of RNA fingerprinting by PCR are theoretically similar but differ in their primer design and application. The most striking difference between differential display and other methods of RNA fingerprinting is that differential display utilizes anchoring primers that hybridize to the poly A tails of mRNAs. As a consequence, the PCR products amplified in differential display are biased towards the 3′ untranslated regions of mRNAs.

The basic technique of differential display has been described in detail (Liang and Pardee, 1992). Total cell RNA is primed for first stand reverse transcription with an anchoring primer composed of oligo dT and any two of the four deoxynucleosides. The oligo dT primer is extended using a reverse transcriptase, for example, Moloney Murine Leukemia Virus (MMLV) reverse transcriptase. The synthesis of the second strand is primed with an arbitrarily chosen oligonucleotide, using reduced stringency conditions. Once the double-stranded cDNA has-been synthesized, amplification proceeds by standard PCR techniques, utilizing the same primers. The resulting DNA fingerprint is analyzed by gel electrophoresis and ethidium bromide staining or autoradiography. A side by side comparison of fingerprints obtained from tumor versus normal tissue samples using the same oligonucleotide primers identifies mRNAs that are differentially expressed.

8. Design and Theoretical Considerations for Relative Quantitative RT-PCR

Reverse transcription (RT) of RNA to cDNA followed by relative quantitative PCR (RT-PCR) can be used to determine the relative concentrations of specific mRNA species isolated from patient tissue. Such determinations would be useful in predicting the susceptibilty of a patienst to diseases such as cancer and gastrointestinal diseases such as Crohn's disease. By determining that the concentration of a specific mRNA species varies, it is shown that the gene encoding the specific mRNA species is differentially expressed. This technique can be used to confirm that mRNA transcripts shown to be differentially regulated by RNA fingerprinting are differentially expressed in the gastrointestinal tract of patients susceptible to GVHD.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

Methods for Increasing or Decreasing MICA or MICB Expression

In one embodiment of the present invention, there are provided methods for the increased gene expression or activation in a cell. This is particularly useful where there is an aberration in the gene product or gene expression is not sufficient for normal function. This will allow for the alleviation of symptoms of biological disorders experienced as a result of allelic mutation in MICA or MICB.

The general approach to increasing gene expression of MICA or MICB in a cell according to the present invention, will be to provide a cell with an MICA or MICB polypeptide. While it is conceivable that the protein may be delivered directly, a preferred embodiment involves providing a nucleic acid encoding an MICA or MICB polypeptide, ie., a MICA or MICB gene, to the cell. Following this provision, the MICA or MICB polypeptide is synthesized by the host cell's transcriptional and translational machinery, as well as any that may be provided by the expression construct. Cis-acting regulatory elements necessary to support the expression of the MICA or MICB gene will be provided, in the form of an expression construct.

In another embodiment of the present invention, there is contemplated the method of blocking the function of MICA or MICB. In this way, it may be possible to curtail any adverse effects of such mutation in a subject. In addition, it may prove effective to use this sort of therapeutic intervention in combination with more traditional therapies for GVHD such as chemosuppression.

The general form that this aspect of the invention will take is the provision, to a cell, of an agent that will inhibit MICA or MICB function. One may employ an antisense nucleic acid that will hybridize either to the MICA or MICB gene or the gene transcript, thereby preventing transcription or translation, respectively. The considerations relevant to the design of antisense constructs have been presented above. Within another aspect one may utilize a MICA or MICB binding protein or peptide, for example, a peptidomimetic or an antibody that binds immunologically to a MICA or MICB the binding of either will block or reduce the activity of the MICA or MICB. The methods of making and selecting peptide binding partners and antibodies are well known to those of skill in the art. Yet another aspect provides the antagonists of MICA or MICB.

Provision of a MICA or MICB gene, a protein, peptide or antagonist, would be according to any appropriate pharmaceutical route. The formulation of such compositions and their delivery to tissues is discussed below. The method by which the nucleic acid, protein or chemical is transferred, along with the preferred delivery route, will be selected based on the particular site to be treated. Those of skill in the art are capable of determining the most appropriate methods based on the relevant clinical considerations.

Where the embodiment involves the use of an antibody that recognizes a MICA or MICB polypeptide, consideration must be given to the mechanism by which the antibody is introduced into the cell cytoplasm. This can be accomplished, for example, by providing an expression construct that encodes a single-chain antibody version of the antibody to be provided. Most of the discussion above relating to expression constructs for antisense versions of MICA or MICB genes will be relevant to this aspect of the invention. Alternatively, it is possible to present a bifunctional antibody, where one antigen binding arm of the antibody recognizes a MICA or MICB polypeptide and the other antigen binding arm recognizes a receptor on the surface of the cell to be targeted.

Advantageously, one may combine this approach with more conventional GVHD therapy options.

Screening For Modulators of MICA or MICB

Within certain embodiments of the invention, methods are provided for screening for modulators of MICA or MICB expression or activity. Such methods may use labeled MICA or MICB proteins or MICA or MICB analogs, anti-MICA or anti-MICB antibodies and the like as reagents to screen small molecule and peptide libraries to identify modulators of MICA or MICB gene expression or MICA or MICB protein activity. Within one example, a modulator screening assay is performed in which cells expressing MICA are exposed to a test substance under suitable conditions and for a time sufficient to permit the agent to effect expression of MICA. The expression of MICA is then detected by incubating the reaction mixture with a MICA specific antibody, which antibody may be labeled directly or may be detected secondarily, (e.g. using a labeled idiotypic or species specific antibody) under conditions that permit the formation of immune complexes between MICA and its specific antibody. The test reaction is compared to a control reaction which lacks the test sample. To complete the modulator screening assay, the presence and/or amount of complexes formed between MICA or MICB and the anti-MICA or MICB antibody is detected in the test sample (e.g. by determining the presence or amount of label bound directly to the antibody or to a secondary antibody directed against the primary antibody). Within this exemplary assay, agents that inhibit expression of MICA or MICB will demonstrate a reduced binding with MICA-specific or MICB specific antibodies relative to the control sample and agents that induce expression of MICA or MICB will demonstrate an increased binding with MICA- or MICB-specific antibodies relative to the control sample.

Within other aspects of the invention, modulator screening assays may be carried out to identify agents that compete with MICA or MICB for binding to a MICA- or MICB-binding molecule (e.g. an antibody or a T-cell receptor). Assays in this context may use whole cells expressing MICA or MICB or use purified truncated MICA or MICB proteins such as those containing only the extracellular domains of the protein (described previously). Within exemplary assays, agents are tested for the ability to compete with MICA or MICB in the context of whole cells or as truncated molecules, for binding to an antibody or T-cell receptor. Thus, within one exemplary assay, a test agent is incubated with a reaction mixtures containing MICA or MICB expressing cells and T-cells bearing MICA or MICB receptors under suitable conditions and for a time sufficient to permit the formation of MICA or MICB-receptor complexes in the absence of the test agent. Antibodies specific for MICA-MICB-receptor complexes may be used to detect the presence or amount of complex present in the test reaction relative to a control reaction lacking the test agent.

Generally the test substance is added in the form of a purified agent, however it is also contemplated that test substances useful within the invention may include substances present throughout the handling of test sample components, for example host cell factors that are present in a cell lysate used for generating the test sample. Such endogenous factors may be segregated between the test and control samples for example by using different cell types for preparing lysates, where the cell type used for preparing the test sample expresses a putative test substance that is not expressed by the cell type used in preparing the control sample.

Yet additional screening methods utilize host cells transformed or transfected with expression constructs incorporating MICA or MICB or encoding MICA or MICB analogs, to provide an in vivo assay mixture. Cells thus transformed, transfected or intracellularly exposed can be used, for example, in screens to detect and identify compounds capable of modulating MICA or MICB function. In this regard, cell lines derived from transgenic animals may be particularly preferred. Immortalized cell lines may be produced, for example, by transformation or transfection with a DNA sequence encoding the SV40 T antigen or with papillomavirus genes E6 and E7 (Kaur and McDougall, 1988; Kaur et al., 1989; Halbert et al., 1991). Alternatively, MICA or MICB expressing cell lines may be generated from MICA or MICB transgenic mice crossed with an IMMORTOMOUSE (Charles River Laboratories, Wilmington, Mass.). IMMORTOMOUSE is a mouse carrying a H-2 Kb-tsA58 SV40 large T antigen transgene. The progeny of the cross are subjected to PCR analysis as generally described above to identify progeny carrying the transgene and are heterozygous for the MICA or MICB gene. The progeny carrying both transgenes (H-2 Kb-tsA58 SV40 large T antigen and MICA or MICB) are back-crossed to the MICA or MICB transgenic mice. The progeny of the back-cross are subjected to PCR analysis to identify mice homozygous for the MICA or MICB transgene carrying the T antigen transgene. Cells from the resulting mice may be immortalized by culturing the cells at 33° C. in the presence of interferon.

Pharmaceutical Compositions

Where clinical applications are contemplated, it will be necessary to prepare pharmaceutical compositions—vectors, MICA- or MICB-expressing cells, MICA or MICB polypeptides, sense and antisense MICA or MICB oligonucleotides or constructs, or modulators of MICA or MICB expression—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics.

Antibodies, ELISAs and Western Blots

1. Generation of Antibodies

In another aspect, the present invention contemplates binding agents that are immunoreactive with MICA, MICB, both MICA and MICB or any portion thereof. Binding agents include polyclonal or monoclonal antibodies and fragements thereof. In a preferred embodiment, an antibody is a monoclonal antibody. Such antibodies may form part of an immunodetection kit as described herein below.

An antibody of the present invention may be a bispecific antibody that is capable of recognizing both MICA and MICB. Multispecificity is a phenomenon that defines the ability of a single antibody molecule to combine with different antigens. Although a single antibody molecule has a unique three dimensional structure it can combine with the inducing antigenic determinant, determinants with similar structures (cross-reacting antigens), and perhaps even determinants with quite disparate structures. A stable antigen-antibody complex will result whenever there is a sufficient number of short-range interactions regardless of the fit. Within the antigen-combining site, a lack of fit in one region can be compensated for by increased binding elsewhere.

Means for preparing and characterizing antibodies are well known in the art (See, e.g., Harlow and Lane, 1988; incorporated herein by reference). More specific examples of monoclonal antibody preparation are given in the examples below. Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

In certain embodiments, the antibodies generated in the animals above may be humanized. Humanized antibodies are of an animal origin that have been modified, using genetic engineering techniques, to replace the constant region and/or variable region framework sequences with human sequences, while retaining the original antigen specificity. Such antibodies are commonly derived from rodent e.g. murine, antibodies with specificity for human antigens and are to be used for in vivo therapeutic applications. This strategy reduces the host response to foreign antibody and allows selection of the human effector functions that are activated. Initially only the Fc regions of human antibodies were substituted but it is now possible to substitute all but the complementarity determining regions of the rodent antibody can be replaced by human sequences. The skilled artisan is referred to Winter and Milstein (1991) for a more detailed description of generation of humanized antibodies.

Antibodies, both polyclonal and monoclonal, specific for the peptides or proteins of the present invention may be prepared using conventional immunization techniques, as will be generally known to those of skill in the art. A composition containing antigenic epitopes of the compounds of the present invention can be used to immunize one or more experimental animals, such as a rabbit, a mouse, a rat, a hamster, a guinea pig, a goat, a pig a horse etc., which will then proceed to produce specific antibodies against the compounds of the present invention. Polyclonal antisera may be obtained, after allowing time for antibody generation, simply by bleeding the animal and preparing serum samples from the whole blood.

It is proposed that the polyclonal and monoclonal antibodies of the present invention will find useful application in standard immunochemical procedures, such as ELISA and Western blot methods and in immunohistochemical procedures such as tissue staining, as well as in other procedures which may utilize antibodies specific to MICA or MICB related antigen epitopes. For example, such antibodies may be employed in antibody cloning protocols to obtain cDNAs or genes encoding MICA, MICB or related proteins. They may also be used in inhibition studies to analyze the effects of MICA or MICB related peptides in cells or animals. Anti-MICA or MICB related antigen antibodies will also be useful in immunolocalization studies to analyze the distribution of MICA or MICB related peptides during various cellular events, for example, to determine the cellular or tissue-specific distribution of the MICA or MICB related peptide under different physiological conditions. A particularly useful application of such antibodies is in purifying native or recombinant MICA or MICB related peptide, for example, using an antibody affinity column. The operation of all such immunological techniques will be known to those of skill in the art in light of the present disclosure.

Additionally, it is proposed that monoclonal antibodies specific to the particular MICA or MICB alleles may be utilized in other useful applications. For example, their use in immunoabsorbent protocols may be useful in purifying native or recombinant MICA or MICB isoforms or variants thereof As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immununogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified MICA or MICB protein, polypeptide or peptide or cell expressing high levels of MICA or MICB. The immunizing composition is administered in a manner effective to stimulate antibody producing cells. Rodents such as mice and rats are preferred animals, however, the use of rabbit, and sheep cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/SXX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as fLPLC or affinity chromatography.

2. The Use of ELISAs and Western Blots to Screen for MICA, MICB and Related Polypeptides

The antibodies of the present invention can be used in characterizing the MICA and MICB content of healthy and diseased tissues by detection of MICA and/or MICB directly on the surface of cells and through techniques such as ELISAs and Western blotting.

The use of antibodies of the present invention, in an ELISA assay is contemplated. MICA, MICB or antigenic sequences derived therefrom are immobilized onto a selected surface, preferably a surface exhibiting a protein affinity such as the wells of a polystyrene microtiter plate. After washing to remove incompletely adsorbed material, it is desirable to bind or coat the assay plate wells with a nonspecific protein that is known to be antigenically neutral with regard to the test antisera such as bovine serum albumin (BSA), casein or solutions of powdered milk. This allows for blocking of nonspecific adsorption sites on the immobilizing surface and thus reduces the background caused by nonspecific binding of antisera onto the surface.

After binding of antigenic material to the well, coating with a non-reactive material to reduce background, and washing to remove unbound material, the immobilizing surface is contacted with the antisera or clinical or biological extract to be tested in a manner conducive to immune complex (antigen/antibody) formation. Such conditions preferably include diluting the antisera with diluents such as BSA, bovine gamma globulin (BGG) and phosphate buffered saline (PBS)/TWEEN. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/TWEEN, or borate buffer.

Following formation of specific immunocomplexes between the test sample and the bound antigen, and subsequent washing, the occurrence and even amount of immunocomplex formation may be determined by subjecting same to a second antibody having specificity for the first. To provide a detecting means, the second antibody will preferably have an associated enzyme that will generate a color development upon incubating with an appropriate chromogenic substrate. Thus, for example, one will desire to contact and incubate the antisera-bound surface with a urease or peroxidase-conjugated anti-human IgG for a period of time and under conditions which favor the development of immunocomplex formation (e.g., incubation for 2 hr at room temperature in a PBS-containing solution such as PBS/TWEEN).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)6-sulfonic acid (ABTS) and H₂O₂, in the case of peroxidase as the enzyme label. Quantitation is then achieved by measuring the degree of color generation, e.g. using a visible spectrum spectrophotometer.

The antibodies of the present invention are particularly useful for the isolation of antigens by immunoprecipitation. Immunoprecipitation involves the separation of the target antigen component from a complex mixture, and is used to discriminate or isolate minute amounts of protein. For the isolation of membrane proteins cells must be solubilized into detergent micelles. Nonionic salts are preferred, since other agents such as bile salts, precipitate at acid pH or in the presence of bivalent cations.

In an alternative embodiment the antibodies of the present invention are useful for the close juxtaposition of two antigens. This is particularly useful for increasing the localized concentration of antigens, e.g., enzyme-substrate pairs.

The compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. This is especially useful when the antigens studied are immunoglobulins (precluding the use of immunoglobulins binding bacterial cell wall components), the antigens studied cross-react with the detecting agent, or they migrate at the same relative molecular weight as a cross-reacting signal.

Immunologically-based detection methods for use in conjunction with Western blotting include enzymatically-, radiolabel-, or fluorescently-tagged secondary antibodies against the toxin moiety are considered to be of particular use in this regard.

3. Immunodetection Kits

In still further embodiments, the present invention concerns immunodetection kits for use with the immunodetection methods described above. As the encoded proteins or peptides may be employed to detect antibodies and the corresponding antibodies may be employed to detect encoded proteins or peptides, either or both of such components may be provided in the kit. The immunodetection kits will thus comprise, in suitable container means, an encoded protein or peptide, or a first antibody that binds to an encoded protein or peptide, and an immunodetection reagent.

In certain embodiments, the encoded protein or peptide, or the first antibody that binds to the encoded protein or peptide, may be bound to a solid support, such as a column matrix or well of a microtiter plate.

The immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the encoded protein or polypeptide antigen, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Characterization of the Expression of a Cell Stress Regulated Human MHC Class I Gene

1. Materials and Methods

DNA clones and constructs, cell lines and transfections. The cDNA hybrid constructs were made in consecutive steps using the primer overlap-extension technique and PCR (Horton et al., 1996), with MICA and mouse H₂-K^{b} and House H₂-D^{b} cDNA templates (Waneck et al., 1987). Error-free cDNA hybrids were identified by sequencing, subcloned into RSV.5neo and stably transfected into C1R cells (Alexander et al., 1990) by electroporation as described (Grandea et al., 1995). Using the same procedure and conditions, lymphoblastoid cell line (LCL) mutants Daudi (Arce-Gomez et al., 1978) and 5.2.4 (Mellins et al., 1991) were transfected with MICA cDNA in RSV.5neo and stable transfectants were selected with G418 (Gibco, 0.5 and 0.1 mg/ml, respectively). Mouse LTK fibroblasts were transfected with cosmid M32A (Spies et al., 1989) using LIPOFECTIN (GIBCO BRL, Gaithersburg, Md.) and were selected with G418 (1 mg/ml). In all transfection experiments, stable positive isolates were identified by indirect immunofluorescence stainings with specific monoclonal antibodies purified from ascites or from hybridoma supernatants and flow cytometry using a FACScan (Becton Dickinson, Immunocytochemistry, San Jose, Calif.) and, when necessary, cloned by limiting dilution. MICA-D^{b} and -K^{b} hybrid molecules on C1R transfectant cells were detected with mABs 28-14-8 (anti-D_{b} α3) and Y3 (anti-K^{b} α1α2), respectively (Ozato and Sachs, 1980, Hammerling et al., 1982). The HT-29 colon carcinoma and U-373 astrocytoma cell lines were from the American Type Culture Collection (Rockville, Md.). Cell lines were grown in RPMI supplemented with 10% fetal calf serum, 10 mM N-2-hydroxyethylpeperazine-N′-2-ethane sulphonic acid (Hepes), 2 mM glutamine and antibiotics.

Production of monoclonal antibodies. RBF/DnJ mice (The Jackson Laboratories; Bar Harbor, Me.) were injected intraperitoneally three times at weekly intervals with 10⁸ C1R cells expressing MICA mRNA after stable transfection with MICA cDNA in RSV.5neo. After a final boost immunization, isolated splenocytes in suspension were fused with P3-X63Ag8.653 myeloma cells (Kearney et al., 1979) by standard polyethylene glycol treatment (Harlow and Lane, 1988). Hybridomas were grown in RPMI media with 10% of each CPSR-3 heat inactivated serum replacement controlled process type 3 (Sigma, St. Louis, Mo.) and Hybridoma Enhancing Supplement conditioned cultured medium from a murine lymphoma cell line (Sigma, St. Louis, Mo.) in 96-well plates under HAT (hypoxanthine aminopterin thymidine) selection on irradiated MRC-5 feeder cells (Harlow and Lane, 1988). Supernatants were differentially screened for specific reactivity with C1R-MICA cells versus untransfected C1R cells by indirect immunofluorescence and flow cytometry. Hybridomas from positive wells were subcloned twice. The isolated mABs 56, 83 and 2C10 are of the IgG2a, IgGl and IgG3 isotypes, respectively.

Labeling, immunoprecipitation and detection of MICA. For surface labeling, washed cells in phosphate-buffered saline (PBS) were biotinylated with Sulfo-NHS-LC-biotin (Pierce Chemical Co., Rockford, Ill.) (100 μg/ml) for 30 min at 4° C. and reactions quenched by addition of 25 mM lysine. 1-3×10⁷ cells were lysed in 1 ml lysis buffer [1% Triton X-100, 50 mM Tris-OH (pH 7.4), 150 mM NaCl, 5 mM EDTA, 5 mM iodoacetamide, protease inhibitors]. Protein in cleared supernatants was quantitated with a MicroBCA kit (Pierce, Chemical Co., Rockford, Ill.) and lysates were precleared using ULTRALINK-Protein A/G beads (Pierce Chemical Co.). MICA was precipitated with purified mAB 56 and protein A/G beads and immunocomplexes washed. Aliquots were treated with N-glycanase (PNGase F, New England Biolabs Inc., Beverly, Mass.) as recommended by the manufacturer. Dissociated and dithiothreitol-reduced immunocomplexes were separated by SDS-polyacrylamide gel electrophoresis (SDS-PAGE) and electroblotted onto nitrocellulose using a Trans-Blot Semi-Dry Transfer Cell (Bio-Rad, Laboratories, Inc., Hercules, Calif.). After overnight incubation of membranes in PBS containing 10% dry nonfat milk, 0.05% TWEEN 20 and 0.02% Na-azide, they were repeatedly washed in TST [0.15 M NaCl, 10 mM Tris-OH (pH 7.4), 0.3% TWEEN 20] and reacted with avidin-horseradish peroxidase (Vector Laboratories Inc., Burlingame, Calif.) in TST (2.5 μg/ml) for 1 hour at 4° C. Membranes washed with TST were treated with ECL enhanced chemiluminescent reagent (Amersham, Life Science, Arlington Heights, Ill.) and exposed to X-ray film. For pulse-labeling and chase, 5×10⁶ cells per time point were labeled with 0.5 mCi [³⁵S]methionine for 5 min. as described (Grandea et al., 1995). For chase, cells were spun through PBS with 10 mM methionine and resuspended in growth media for the indicated time periods. Cells were lysed, and MICA protein was precipitated using mAB 2C10 as described above. Isolated and denatured MICA was treated with endoglycosidase H (Endo H, New England Biolabs) as recommended by the manufacturer and analysed by SDS-PAGE. Fixed gels were treated with AMPLIFY (Amersham) and dried for autoradiography.

Tissues and Immunohistology. Tissue samples from autopsies, biopsies or surgical specimens were embedded in TISSUE-TEK II O.C.T compound, a specimen matrix for cryostat sectioning (VWR Scientific Products, West Chester, Pa.) and frozen in liquid N₂-precooled methylbutane. Cryostat 4 μm sections mounted on poly L-lysine-coated slides were air-dried, fixed in cold acetone and overlaid with purified primary mAB appropriately diluted in staining buffer (PBS with 2% goat and 2% human serum, 0.1% TWEEN-20). After overnight incubation at 4° C., slides were washed and overlaid with fluorescein-conjugated goat F(ab′)₂ anti-mouse IgG (Tago Inc., Burlingame, Calif.) diluted 1:150 in staining buffer. Coverslipped sections were examined by confocal immunofluorescence microscopy. For the double-stainings nuclei were visualized with propidium iodide. The epithelial desmosomal cadherin desmoglein-I was detected with biotinylated mAB DG3.10 (Schmidt et al., 1994) (Progen, Heidelberg, Germany) and Texas Red-conjugated streptavidin as the second layer. Autopsy tissue specimens included brain, heart, lung, thyroid, liver, kidney, skin, adrenal gland, placenta, tonsil and spleen and were obtained from Swedish Hospital (Seattle, Wash.). Gastric and small and large intestinal biopsy specimen were kindly provided by D. Levine (University of Washington Medical Center, Seattle, Wash.). Thymus specimens from corrective infant cardiac surgery were kindly provided by D. Hall and F. Lupinetti (Children's Hospital, Seattle, Wash.). For antibody staining and flow cytometry analysis, thymocyte suspensions were prepared by passing minced tissue through wire mesh. Peripheral blood mononuclear cells from randomly selected donors were isolated by density gradient centrifugation through Ficoll-Hypaque (Pharmacia).

Promoter mapping and sequencing, RNA blot hybridization and heat shock induction. MICA and MICB exon 1 and promoter sequences were mapped to 5.9 kilobases (kb) Xho I/Kpn I and 0.6 kb Bam HI/Bgl II fragments of the cosmids M32A and R5A, respectively, by blot hybridization with a [³²P]dCTP-labeled exon 1-derived oligonucleotide (Bahram et al., 1994). Sequences were obtained by dideoxynucleotide chain-termination from the double-stranded templates using the exon 1 oligonucleotide and upstream primers, [3S]dATP and SEQUENASE, T7 DNA polymerase (United States Biochemical, Cleveland, Ohio). RNA extractions, electrophoresis (20 μg/lane), blot transfers and hybridizations with [³²P]dCTP-labeled DNA probes were according to standard protocols (Sambrook et al., 1989). The HLA-B-specific probe was the 885 base-pair (bp) Xba I fragment from the genomic clone B7-14 (Barbosa et al., 1982). The human HSP70 probe was a 2.3 kb Bam HI/Hind III genomic DNA fragment (Wu et al., 1985). For heat shock treatment, HeLa cells grown in petri dishes were floated on a 42° water bath for various periods of time.

2. Results and Discussion

The substantial divergence of the MICA amino acid sequence translated from cloned genomic and corresponding complementary DNA (cDNA) from all class I sequences indicated that none of the available anti-class I antibody reagents could be expected to recognize this putative gene product (Bahram et al., 1994). The inventors initially chose an indirect approach to monitor expression of MICA, by transfecting C1R cells with hybrid cDNA constructs in which all domain sequences of MICA were variously substituted with corresponding mouse H2-K^{b} or -D^{b} sequences encoding epitopes recognized by defined monoclonal antibodies (mABs).

Indirect immunofluorescence stainings with the mABs Y3 (anti-K^{b} α1α2) or 28-14-8 (anti-D^{b} α3) (Hammerling et al., 1982) and flow cytometry revealed that all of the epitope-tagged hybrid molecules were present at substantial levels on the surfaces of the stably transfected cells. This indicated that the MICA cDNA encoded a class I-like membrane-anchored cell surface protein, thereby providing the necessary rationale for the further investigation of the expression of MICA. To obtain reagents suitable for diverse experimental applications, specific mABs were generated after immunizing mice with C1R cells transfected with the MICA cDNA and differentially screening hybridoma supernatants by indirect immunofluorescence and flow cytometry. The isolated mABs 56, 83 and 2C10 bound to C1R-MICA transfectant cells but not to untransfected C1R cells and were not cross-reactive with putative MICB on multiple independent C1R-MICB cDNA transfectants. The indirect immunofluorescence studies showed that large amounts of MICA were present on the surfaces of C1R-MICA, LTK-MICA, mutant 5.2.4-MICA (TAP⁻) and Daudi-MICA (β₂m⁻) cells, as well as on HT29 colon carcinoma and U373 astrocytoma cell lines.

To analyse MICA molecules, immunoprecipitations were performed with mAB 56 from lysates of surface-biotinylated C1R-MICA and C1R control cells. Samples were treated with N-glycanase or left untreated. After SDS-polyacrylamide gel electrophoresis (SDS-PAGE) and transfer onto nitrocellulose, biotin-labeled protein was detected with avidin-peroxidase and chemiluminescent reagent. With N-glycanase digestion, a single compact band of 43 kDa was evident from C1R-MICA transfectants, which was missing in the C1R negative control and matched the predicted molecular mass of the MICA polypeptide (Bahram et al., 1994). It showed the same electrophoretic mobility as glycosylated HLA-C isolated with mAB W6/32 from C1R-MICA (HLA-A⁻, B⁻) cell lysate (Alexander et al., 1990, Parham et al., 1979). Without N-glycanase digestion, the inventors detected a broad band in the 65-75 kDa molecular mass range. Thus, bona fide MICA molecules were expressed on the surfaces of C1R-MICA cells and were highly glycosylated, consistent with the 8 potential N-glycosylation acceptor sites in the α1α2α3 domain sequences of MICA (Bahram et al., 1994). This distinguished MICA from all conventional and nonclassical MHC class I molecules, which have a single glycosylation site at position 86 in the al domain that is not conserved in MICA (Bjorkman and Parham, 1990; Bahram et al., 1994). Similar results were obtained using mABs 83 and 2C10, of which the former reacted less efficiently in these experiments, and with mouse LTK cells transfected with a cosmid encoding the MICA gene.

The subunit composition and transport of MICA molecules was investigated using C1R-MICA cells and mutant lymphoblastoid cell line Daudi (β₂M) and 5.2.4 (TAP⁻(Arce-Gomez et al., 1978, Mellins et al., 1991) cells transfected with the MICA cDNA. Immunoprecipitation with the anti-human β₂M mAB BBM1 (Parham et al., 1983) coprecipitated HLA-C but not MICA from lysates of surface-labeled C1R-MICA cells. Moreover, C1R-MICA and untransfected C1R cells showed the same fluorescence intensity after staining with mAB BBM1 and Daudi-MICA cells displayed large amounts of surface MICA, as observed by staining with mAB 56 and by immunoprecipitation of surface-labeled MICA molecules. In further support of these results, no β₂M was coprecipitated with MICA from lysates of metabolically labeled C1R-MICA cells using mAB 2C10, and newly synthesized MICA molecules were transported through the Golgi and matured at similar rates in C1R-MICA and Daudi-MICA cells, as determined by metabolic pulse-labeling and chase and by treatment of immunoprecipitated proteins with endoglycosidase H before SDS-PAGE. A comparison of immunoprecipitated protein treated with endoglycosidase H and untreated immunoprecipitated protein was carried out to measure the acquisition of cleavage-resistant complex-type carbohydrates during transport in Golgi vehicles. Thus, unlike all other MHC class I and related molecules, MICA molecules were not associated with β₂M. Moreover, normal surface levels of MICA were also observed with 5.2.4-MICA cells, which lack the TAP peptide transporter that supplies the peptides mainly bound by class I molecules from the cytosol into the lumen of the endoplasmic reticulum (Mellins et al., 1991, Spies et al., 1990, Lehner and Cresswell, 1996). Thus, distinct from most conventional MHC class I molecules, the transport and apparently stable surface expression of MICA was independent of cytosolic peptide ligands—a characteristic shared with the mouse nonclassical MHC class I T10^{b}, T22^{b} and TL molecules as well as with human CD1b and CD1d (Schild et al., 1994, Weintraub et al., 1994, Kaliyaperumal et al., 1995, Holcombe et al., 1995).

These results and the previously observed restricted transcription of the MICA gene in epithelial cell lines raised the question of the tissue distribution of MICA (Bahram et al., 1994). Indirect immunofluorescence stainings using mABs 56 and 83 confirmed the complete absence of MICA on the surfaces of B-, T- and monocyte cell lines and on freshly isolated peripheral blood mononuclear cells and thymocytes. Variable amounts of MICA were detected on epithelial tumor cell lines such as HT29 colon carcinoma and U373 astrocytoma cells, as indicated by antibody binding and flow cytometry and by immunoprecipitation of the surface-labeled protein. On some epithelial cell lines, surface MICA was low or undetectable despite the presence of similar amounts of MICA mRNA in all of the cell lines tested (Bahram et al., 1994). This discrepancy may suggest that the posttranslational processing and transport of MICA molecules was dependent on factors that were limited or missing in these cell lines and that were different from those required for the normal processing of conventional class I molecules (Lehner and Cresswell, 1996).

The tissue distribution of MICA was examined by immunofluorescence microscopy of mAB 83 and FITC-goat F(ab′)₂ anti-mouse IgG-stained cryosections. Most of the tissues were negative for binding of mAB 83. These included brain, heart, lung, thyroid, liver, kidney, skin, adrenal gland, placenta, tonsil and spleen. Strongly positive staining was seen in gastric and in small and large intestinal mucosa biopsies from several individuals and was confined to surface and glandular epithelial cells. In intestinal epithelium, fluorescent cells lined the villi but were not apparent in the undifferentiated crypts. Notably, the observed staining was often discontinuous, with variably sized interspersed areas of nonfluorescent epithelium, which occasionally included single fluorescent cells. IgG1 matched-isotype controls and stainings of dog intestinal mucosa sections gave negative results. In specimens of infant thymus, mAB 83 stained a population of stellate cells in the subcapsular cortex, which were epithelial cells because they were positive for the desmoglein-I epithelial cell marker (Schmidt et al., 1994).

The remarkably restricted and varied expression of MICA in intestinal epithelium suggested an unusual transcriptional regulation of the MICA gene. 5′-end flanking region sequences of MICA and MICB upstream of their translation initiation codons were derived from mapped cosmid fragments. Both sequences were unrelated to the transcriptional control regions of conventional class I genes. They contained heat shock elements defined in HSP70 genes and shared further nucleotide sequence homologies with the human HSP70 promoter (Hunt and Morimoto, 1985). Notably, MICA and MICB are closely linked to HSP70 genes in the MHC (Sargent et al., 1989, Spies et al., 1989).

The HSP70 sequence contains a consensus heat shock element that is almost perfectly matched by MICA and MICB promoter sequences (Hunt and Morimoto, 1985, Morimoto, 1993). Increased steady-state levels of MICA and MICB mRNA in Hela cells after heat shock, as indicated by RNA blot hybridizations with a labeled MICA cDNA probe. MICB mRNA of 2.4 kb is larger than MICA mRNA due to a longer 3′-untranslated region (Bahram and Spies, 1996). Heat shock induction at 42° C. for increasing periods of time and kinetics of induction after a 1-hour exposure to 42° C. were studied. In both experiments, the kinetics of HSP70 mRNA induction were very similar. Blots hybridized with the HSP70 probe were exposed to film for much shorter time. Control HLA-B mRNA remained unaltered in these experiments.

Exposure of Hela cells to heat shock resulted in moderate and substantial increases of the steady-state levels of MICA and MICB mRNA, respectively, with induction kinetics that were similar to data obtained in parallel for HSP70 mRNA as measured by RNA blot hybridization with a labeled MICA cDNA probe control HLA-B mRNA remained unaltered. Because these results were obtained with rapidly proliferating tissue culture cells in which MICA but not MICB is constitutively transcribed at a significant rate (Bahram et al., 1994), they may quantitatively underestimate the induction of MICA mRNA in quiescent gastrointestinal epithelial cells in which the MICA gene appeared not to be constitutively expressed. This deregulated transcription of the MICA gene in epithelial cell lines with resultant intermediate or high surface expression of MICA and the only moderate mRNA induction may at least partially explain why the inventors repeated attempts with various cell lines to detect increased surface levels of MICA after heat shock treatment were unsuccessful at the time. This was however susequently shown as described herein below. Because heat shock is one condition that triggers the general cell stress response, the results argue that MICA and more obviously MICB are cell stress response genes (Craig and Gross, 1991, Morimoto, 1993).

In summary, these results demonstrate the surface expression of a previously unrecognized human MHC class I molecule with unusual structural and biological characteristics. The expression of MICA without associated β₂M, independent of TAP and almost exclusively in gastrointestinal epithelium is unique among all class I-like molecules although partial similarities exist among the mouse nonclassical MHC class I T10^{b}, T22^{b} and TL, molecules and human CD1b and CD1d (Schild et al., 1994, Weintraub et al., 1994, Kaliyaperumal et al., 1995, Porcelli et al., 1992, Hanau et al., 1994, Balk et al., 1994).

Unlike certain class I-related molecules that are encoded outside the MHC and have conserved functions not associated with T-cell recognition (Araki et al., 1988, Simister and Mostov, 1989), the characteristics of the MHC class I MICA and putative MICB molecules imply that these are recognized by a subset of T-cells in gastrointestinal epithelium in an interaction that is probably independent of conventional MHC class I antigen processing. This is supported by the expression of MICA in thymic cortical epithelial cells, suggesting a possible role of MICA in T-cell selection, and by the existence of a minimum of 16 allelic variants with clustered amino acid substitutions in the α1α2α3 domains of MICA (Bahram et al., 1994). Because the expression of MICA and putative MICB is to some degree coupled to cell stress, these molecules could be recognized alone or possibly complexed with ligands derived from heat shock proteins.

In human intestinal epithelium, the mono- or oligoclonal expansion of T-cells with γδ (Vol) or αβ T-cell receptors are unexplained phenomena (Deusch et al., 1991, Van Kerckhove et al., 1992, Chowers et al., 1994, Gross et al., 1994). However, stimulation of these T-cells by autologous antigens that may be stress-induced has been postulated (Van Kerckhove et al., 1992, Gross et al., 1994, Kagnoff, 1993). Thus, MICA and possibly MICB may represent a ‘missing link’ in the gut mucosal immune system.

### Example 2

### Generation of Transgenic Animals

Transgenic mice expressing the human MICA gene were produced as generally described in “Manipulating the Mouse Embryo; A Laboratory Manual” 2nd edition (eds., Brigid Hogan, Rosa Beddington, Frank Costantimi and Elizabeth Long, Cold Spring Harbor Laboratory Press, 1994; which is incorporated herein by reference in its entirety). To induce superovulation, 3 week-old (C57/BL6×SJL)F₁/J hybrid female mice were injected with pregnant mare's serum (PMS) and human chorionic gonadotropin (hCG). Pregnant mare's serum (Sigma, St. Louis, Mo.) was resuspended to 50 IU/ml in sterile 0.9% sodium chloride. The preparation was aliquoted and stored at −20° C. Human chorionic gonadotropin (Sigma, St. Louis, Mo.) was resuspended in sterile water to 500 IU/ml, aliquoted into 100 microliter aliquots, lyophilized and stored, light protected, at −20° C.

Prior to administration, the hormone was resuspended in 0.9% sodium chloride for injection. Each mouse was injected intraperitoneally with 5 IU of PMS. After a 42 to 48 hour interval, the mice were then injected intraperitoneally with 5 IU of hCG. The mice were mated with (C57/BL6×SJL)F₁/J hybrid males after injection with hCG, and eggs at the one-cell stage were harvested the following morning. The eggs surrounded by Cumulus cells were isolated manually from dissected oviducts into a solution containing 300 microgram per milliliter of hyaluronidase in M2 medium (94.66 mM NaCl, 4.78 mM KCl, 1.71 mM CaCl₂.2H₂O, 1.19 mM KH2PO₄, 1.19 mM MgSO₄.7H₂O, 4.15 mM NaHCO₃, 20.85 mM HEPES, 23.28 mM sodium lactate, 0.33 mM sodium pyruvate, 5.56 mM glucose, 4 g/l BSA, 0.06 g/l penicillin G-potassium salt, 0.05 μl streptomycin sulfate, 0.01 g/l Phenol Red). The cells were incubated in the hyaluronidase solution until the Cumulus cells fell away from the eggs. The eggs were transferred using sterile transfer pipettes into fresh M2 medium to remove excess hyaluronidase solution. After this wash, the eggs were transferred to M16 (94.66 mM NaCl, 4.78 mM KCl, 1.71 mM CaCl₂.2H₂O, 1.19 mM KH₂PO₄, 1.19 mM MgSO₄.7H₂O, 25 mM NaHCO₃, 20.85 mM HEPES, 23.28 mM sodium lactate, 0.33 mM sodium pyruvate, 5.56 mM glucose, 0.06 g/l penicillin G-potassium salt, 0.05 g/l streptomycin sulfate, 0.01 g/l Phenol Red) for culture at 37° C. until needed.

For microinjection, several eggs were transferred to an injection chamber, and the eggs were visualized to identify eggs at containing pronuclei. Each egg was manipulated onto the tip of the holding pipet to permit injection into the pronucleus. The tip of the injection pipetted was filled with 2.5 nanograms per microliter of the DNA solution. The DNA used for microinjection was derived from a cosmid clone containing the complete MICA gene flanked by genomic DNA sequences. The MICA cosmid clone was isolated from a genomic DNA sequences. The MICA cosmid clone was isolated from a genomic cosmid library prepared from peripheral blood mononuclear cells from a random individual. The MICA-containing cosmid was isolated using a labeled MICA cDNA probe using standard conditions. The presence of the complete MICA gene was confirmed by mapping with restriction enzymes and comparison with a MICA restriction map and sequence.

For microinjection, a 23 kb fragments containing the MICA gene was obtained by cleavage with Xba I and Sal I followed by purification to isolate the fragments from adjacent genomic DNA and from cosmid vector derived sequences. The injection pipette was inserted into the egg, through the zona pellucida, and the DNA was injected into the pronucleus. The procedure was repeated until several hundred eggs were microinjected. The healthy injected eggs were separated, placed in culture medium and incubated at 37° C. overnight to permit the eggs to progress to the two-cell stage.

The injected two-cell stage eggs were transferred to the oviducts of 0.5-day post-coitus pseudopregnant female B6CBA F1 mice. The embryos were transferred to M2 medium before loading into the transfer pipet Fifteen embryos in a minimal volume of M2 medium were loaded into a transfer pipette. The embryos were transferred into the surgically-exposed opening of the oviduct of the pseudopregnant mouse. The embryos are permitted to develop to term.

Tail biopsies of the resulting F2 mice were obtained and screened for integration of the transgene by PCR amplification of a 850 bp genomic DNA fragment encompassing exons 3 and 4 of the MICA gene. Positive founder mice were bred with (C57/BL6×SJL)F1/J hybrids. In each litter, mice were tested for stable germline transmission of the MICA gene using PCR amplification of the 850 bp genomic DNA fragment encompassing exons 3 and 4, described above. To detect productive expression of the MICA gene, selected mice were sacrificed, and their intestines were removed and tested for the presence of MICA mRNA by reverse-transcription PCR (RT-PCR) with purified RNA and 25-mer oligonucleotides derived from the beginning an end of the MICA open reading frame. Gut tissue samples from several mice produced MICA RT-PCR cDNA fragments of the predicted 1200 bp length.

### Example 3

### Allelic Repertoire of the Human MHC Class I MICA Gene

The present Example illustrates allelic variants of MICA, these variants are only exemplary and the methods described herein may be used to isolate further variants of MICA or MICB.

The hallmark of the classical major histocompatibility complex (MHC) class I molecules is their astonishing level of polymorphism, a characteristic not shared by the nonclassical MHC class I genes. A distinct family of MHC class I genes has been recently identified within the human MHC class I region. The MICA (MHC class I chain-related A) gene in this family is a highly divergent member of the MHC class I family and has a unique pattern of tissue expression. The inventors have sequenced exons encoding the extracellular α1, α2, and α3 domains of the MICA gene from twenty HLA homozygous typing cell lines and four unrelated individuals. The inventors report the identification of eleven new alleles defined by a total of twenty-two amino acid substitutions. Thus, the total number of MICA alleles is sixteen. Interestingly, a tentative superimposition of MICA variable residues on the HL4-A2 structure reveals a unique pattern of distribution, concentrated primarily on the outer edge of the MICA putative antigen binding cleft, apparently bordering an invariant ligand binding site.

Materials and Methods

Genomic DNA

Genomic DNA from eighteen HLA homozygous typing cell lines (HTCLs) and four unrelated individuals were used in this investigation. The HTCLs studied were the following: KAS116, JESTHOM, AMA1, SCHU, MGAR, RML, DUCAF, SAVC, BMIS, AMALA, LWAGS, BM92, LKT3, PF97387, WT49, AKIBA, LB, and JY (Table3). High relative molecular mass genomic DNA from four unrelated individuals was isolated from peripheral blood leukocytes using the Q1AMP blood kit (Otagen, Hilden, Germany). The nucleotide sequence of the MICA gene in MANN (Bahram et al., 1996) and CGM1 (Fodil et al., 1996) cell lines were also included in the final analysis.

TABLE 3
MICA alleles within HLA haplocyes. All cells are HTCLs except GGMI.
The MICA alleles of EI32B, YAR, MOU, and PITOUT are from Bahram
and co-workers (1994). The MANN sequence has been previously
published (Bahram et al., 1996) and, finally, the CGMI allele
was obtained from sequence determination of a continuous segment
of DNA linking MICA to HLA-C (Mizuki and co-workers).

| HTCL | MICA | B | Cw | A |
|---|---|---|---|---|
| DUCAF | 001 | 18 | 5 | 30 |
| E132B | 001 | 18 | 5 | 30 |
| YAR | 002 | 38 | — | 26 |
| AMAJ | 002 | 53 | 4 | 28 |
| WT49 | 002 | 58 | — | 2 |
| PITOUT | 003 | 44 | — | 29 |
| MOU | 004 | 44 | — | 29 |
| BM15 | 004 | 49 | 7 | 1 |
| PF97387 | 004 | 44 | — | 29 |
| MANN | 004 | 44 | — | 29 |
| KAS116 | 006 | 51 | — | 24 |
| JESTHOM | 007 | 27 | 1 | 2 |
| BM92 | 007 | 51 | 1 | 25 |
| CGM1 | 008 | 8 | 7 | 3 |
| SCHU | 008 | 7 | 7 | 3 |
| MGAR | 008 | 8 | 7 | 26 |
| SAVC | 008 | 7 | 7 | 3 |
| LB | 008 | 60 | 2/3 | 68 |
| JY | 008 | 7 | 7 | 2 |
| RML | 009 | 51 | — | 2 |
| AKTBA | 009 | 52 | — | 24 |
| AMALA | 010 | 62 | 9 | 2 |
| LWAGS | 011 | 14 | 8 | 33 |
| LKT3 | 012 | 54 | 1 | 24 |

Polymerase chain reaction (PCR™) amplification and sequence determination of MICA gene MICA allelic variants were identified by sequencing of cloned PCR-amplified exons 2-4 by following the previously described protocol (Bahram et al., 1994) or by direct sequencing of PCR-amplified individual exons. For the latter strategy, a 2201 base pair (bp) MICA gene segment encoding the α1, α2, and α3 exons was amplified using the following oligonucleotides (all nucleotide positions are from Bahram et al., 1996): 5′-CGTTCTTGTCCCTTTGCCCGTGTGC-3′ (residues 6823-6847; SEQ ID NO:9) and 3′-AACCCTTCCCTTACCCCCGTCGTAG-5′ (9023-8999; SEQ ID NO:10). The reaction was performed using the EXPAND Long Template PCR System (Boehringer Mannheim, Germany), according to the manufacturer's specifications except that the annealing temperature was 63° C. The second PCR reaction was performed in a total volume of 50 μl containing 2 μl of the previous amplification reaction, using standard Taq DNA Polymerase (Boehringer Mannheim, Mannheim, Germany) and employing pairs of oligonucleotides harboring M13 Forward and Reverse sequences (underlined) at their 5′ termini. These included 5′-TATGTAAAACGACGGCCAGTTTCACCTGTGATTTCCTCTTCCCCA-3′ (6924-6948; SEQ ID NO:11) and 3′-GGTCTTTTCAATCCCCGTCTCTCGTCCAGTATCGACAAAGGACAT-5′ (7270-7246; SEQ ID NO:12) for the α1 exon, 5′-TATGTAAAACGACGGCCAGTTTCGGGAATGGAGAAGTCAC-3′ (7443-7462; SEQ ID NO:13 and 3′-CGAGAGGAGAGGGAGGTTAACCAGTATCGACAAAGGACAT-5′ (7805-7786; SEQ ID NO: 14) for the α2 exon and finally 5′-TATGTAAAACGACGGCCAGTGTTCCTCTCCCCTCCTTAGA-3′ (8294-8313 SEQ ID NO:15) and 3′-AAAAAGTCCCTTTCACGACCACCAGTATCGACAAAGGACAT-5′ (8744-8724; SEQ ID NO:16 for the α3 exon. The PCR products were purified using the QIAquick PCR Purification Kit (QIAGEN. Germany) and sequenced using ABI PRISM Dye Printer Cycle Sequencing Ready Reaction Kit with AMPLITAQ DNA Polymerase. FS (Perkin Elmer, Norwalk, Conn.) according to the manufacturer's protocol. The reactions were run on an ABI 377 DNA Sequencing System and the results were analyzed using ABI Sequence Analysis and Navigator Softwares. All heterozygous positions found on electrophenograms were individualized by subsequent sequencing of cloned DNA fagments (TA Cloning Kit; Invitrogen, San Diego, Calif.) using exon-specific primers (Bahram et al., 1994) and the ABI PRISM Dye Terminator Cycle Sequencing Kit (Perkin Elmer). All allelic sequences reported in this publication have been generated as a result of at least two independent PCR reactions.

Results

Initial work on the extent of MICA polymorphism was limited to four HTCLs and two cDNA clones isolated from two different libraries (Bahram et al., 1994). This work established the existence of five alleles in the MICA gene created by 18 nucleotide substitutions of which 14 were nonsynonymous. Further work was required to fully assess the extent of MICA variability in the population.

In the study reported here DNA from 20 HTCLs and four HLA heterozygous individuals were selected with the purpose of covering a diverse range of HLA haplotypes. Nucleotide sequence determination of MICA exons 2, 3, and 4 was performed either by the sequencing of cloned PCR fragments as previously described (Bahram et al., 1994) or by the direct sequencing of PCR-amplified material. This led to identification of eight nonsynonynous nucleotide exchanges among a total of nine new nucleotide substitutions (Table 4). In conjunction with previous work (Bahram et al., 1994), of 27 nucleotide variations in the coding sequence of MICA, 22 are nonsynonymous (4/6 nucleotide in α), 10/10 in α2, and 8/11 in the α3) and collectively define 11 new MICA alleles, establishing a total of 16 MICA allelic variants (Table 4).

TABLE 4
MICA nucleotide variation. Positions refer to amino acid residues.
Transmembrane (exon 5) sequences have been reported by Mizuki
and co-workers (submitted).

| | Position | Codon | Amino Acid |
|---|---|---|---|
| Exon 2 (α1) | | | |
| | 6 | CGT→CCT | Arg→Pro |
| | 14 | TGG→GGG | Trp→Gly |
| | 23 | CTC→CTT | Leu→Leu |
| | 24 | ACT→GCT | Thr→Ala |
| | 36 | TGT→TAT | Cys→Tyr |
| | 64 | AGA→AGG | Arg→Arg |
| Exon 3 (α2) | | | |
| | 114 | GGG→CGG | Gly→Arg |
| | 122 | CTG→GTG | Leu→Val |
| | 125 | AAG→GAG | Lys→Glu |
| | 129 | ATG→GTG | Met→Val |
| | 151 | ATG→GTG | Met→Val |
| | 156 | CAC→CTC | His→Leu |
| | 173 | AAA→GAA | Lys→Glu |
| | 175 | GGC→AGC | Gly→Ser |
| | 176 | GTA→ATA | Val→Ile |
| | 181 | ACA→AGA | Thr→Arg |
| Exon 4 (α3) | | | |
| | 191 | AGC→AGT | Ser→Ser |
| | 198 | ATT→ATC | Ile→Ile |
| | 205 | TCT→TCC | Ser→Ser |
| | 206 | GGC→AGC | Gly→Ser |
| | 210 | TGG→CGG | Trp→Arg |
| | 213 | ACA→ATA | Thr→Ile |
| | 215 | AGC→ACC | Ser→Thr |
| | 221 | GTA→CTA | Val→Leu |
| | 251 | CAA→CGA | Gln→Arg |
| | 268 | AGC→GGC | Ser→Gly |
| | 271 | CCT→GCT | Pro→Ala |
| Exon 5 (TM) | | | |
| | 293 | (GCT) | (Ala) |
| | 295 | GCT→GGCT | (Ala)_{2...}Stop |
| | | | at 310 |

The MICA polymorphic residues are distributed throughout the extracellular portion of the molecule, but are slightly more prevalent in the α2 domain (ten variants were identified in this domain as compared with four in the α1 and eight in the α3 domain). Among the amino acid substitution, several are drastic in nature and may have radical effects on putative ligand/receptor binding. These are predominantly in the α1 and α2 domains and include R6P, G114R, K125E, H156L, K173E, T181R, W210R, and Q251R. In contrast, six of eight changes in the α3 domain are conservative and among these three are shared by at least one human or mouse class 1 molecule (Bahram et al., 1994). These latter include G206S, T2131, and S215T. It is remarkable that almost none of the MICA variable residues coincide with the polymorphic positions of MHC class I molecules. In fact, a tentative superimposition of MICA polymorphic residues upon the HLA-A2 three-dimensional structure shows that most MICA variants tend to map to the periphery of the putative antigen binding cleft. Thus, the putative ligand binding site may be invariant, perhaps facilitating presentation of a conserved antigen by the MICA molecule. This is radically different from the well-defined pattern of HLA polymorphism which is focused on residues in direct contact with the peptide or the TCR, located in the a l domain-encoded α helice and the α2 domain-encoded β strands (Bjorkman and Parham, 1990). It is also noted that except for G114R none of the MICA variable residues affect residues conserved throughout vertebrate evolution (Bahram et al., 1994) and except for one (S268G) none of these variable residues coincide with the putative N-linked glycosylation sites. It is also noteworthy that nine of the MICA variable residues are located precisely at the positions at which MICA and MICB sequences differ and, indeed, eight of the MICA variants are identical to MICB residues at these positions: T24A, C36Y, K125Q, M129V, G206S, W210R, S215T, and S268G (Bahram and Spies, 1996). This implicates intergenic conversion events as a mechanism for the establishment of MICA polymorphism, as it has been for other HLA molecules. The creation of an MICB-identical patch encompassing residues 206-215 MICA003-006 and 009 could be considered a visible sign of this likely phenomenon.

Finally, a preliminary analysis of MICA alleles within the panel of available MHC haplotypes reveals, a considerable degree of linkage disequilibrium with alleles of the closely linked HLA-B, C, and A genes (Table 3). In this respect, the preferential segregation of MICA001 with HLA-B818, CwS, and A30, MICA004 with HLA-B44, A29, and finally MICA008 with haplytypes carrying HLA-B7, Cw7, and A3 or A2 (Table 3). This tight degree of linkage necessitates formal examination of the involvement of MICA alleles in the MHC-associated diseases (Dausset and Svejgaard, 1977).

### Example 4

### Recognition of Stress-Induced MHC Class I-Related Molecules by Intestinal Epithelial γδ T Cells

To explore a functional relationship, the inventors established T cell lines from lymphocytes extracted from an intestinal epithelial tumor (Groh et al., 1996). Other adequate sources of intestinal epithelium are generally unavailable.

Materials and Methods

Bulk Lymphocytes Extraction and Culture of T-cell Lines

Bulk lymphocytes extracted from surgical specimens of intestinal epithelial tumors were obtained from Dr. K. Grabstein and Dr. H. Secrist (Corixa Corp, Seattle, Wash.). These were prepared by enzymatic digestion of minced tissue samples, sieving through wire mesh, and centrifugation in differential Ficoll-Hypaque (Pharmacia, Piscataway, N.J.) gradients (Whiteside et al., 1986; Ebert, 1989). Collected and washed lymphocytes were seeded in 96-well round bottom plates (10³/well) and cultured in the presence of γ-irradiated C1R-MICA or C1R-MICB cells (2×10⁴ well) in RPMI media supplemented with 8% fetal calf serum, 2% pooled human serum, rhIL-2 (2 IU/ml; Cetus), rhIL-7 (10 ng/ml; obtained from Dr. N. Vita, Sanofi Recherche, France) (Watanabe et al., 1995) phytohaemagglutinine (PHA, 0.5 μg/ml; Difco), glutamine and antibiotics. After one week, pooled lymphocytes were sorted for Vδ1 γδ T cells using fluorescein-isothiocyanate (FITC)-conjugated mAB δTCS1 (Engogen, CITY, STATE) (Wu et d., 1988) and a FACS VANTAGE cell sorter (Becton Dickinson, Lincoln Park, N.J.), and cultured as above with weekly restimulations. Double staining with mAB δTCS1 and biotinylated anti-TCR-γ/δ-1 (Becton Dickinson, Lincoln Park, N.J.) (Borst et al., 1988) indicated that all γδT cells expressed Vδ1. The remainder lymphocytes were αβ T cells, by staining with anti-TCR-α/β-1 (Becton Dickinson, Lincoln Park, N.J.) (Tax et al., 1983), and were grown under identical conditions. T cell lies were first tested 3 weeks after sorting. The δ1A and δ1B lines were CD4⁻ and CD8⁻, as determined with mAB Leu-3a and Leu-2a (Becton Dickinson, Lincoln Park, N.J.), respectively, and α^{E}β₇⁺ by staining with mAB CD103 (Immunotech, CITY, STATE). After 3 more weeks, T cells were grown m the additional of irradiated allogeneic peripheral blood mononuclear cells. T cell clones from the δ1A and δ1B lines were derived after the fist week of culture by limiting dilution plating and expanded for at least 3 weeks before functional testing

Transfection of Cells

MICA cDNA transfectants of C1 R, Daudi and 5.2.4 have been described above and by Groh et al., (1996). Transfections of E14 and Daudi-β₂m cells (Sadasivan et al., 1996) with MICA cDNA (Bahram et al., 1994) in RSV.5neo or in pREP8 [symbol] (Groh et al., 1996; Arnold et al., 1992), and of C1R cells with MICB cDNA (Bahram and Spies, 1996) in RSV.5neo were done as described (Grandea III et al., 1995). Stable transfectant isolates had large amounts of surface MICA or MICB by indirect immunofluorescence stainings with mAB 2C10 or 6D4 and flow cytometry (Example 1, Groh et al., 1996).

Chromium Release Assays

Chromium release assays were according to stand conditions, in 96-well round bottom plates, with target cells plated at 3×10³ per well after labeling for 2 h with ⁵¹Cr]sodium chromate and washing. Adherent target cells were harvested with non-enzymatic cell dissociation solution (Sigma, St Louis, Mo.). Effectors were added at the indicated ratios, briefly centrifuged, and released radioactivity was determined afler 4 h at 37° C. using Luma Plates and a TopCount microplate scintillation counter (Packard). Specific lysis (in %) was calculated using the standard formula [(experimental —spontaneous release/total—spontaneous release) x100]. All experiments were repeated least once and in most cases several ties. For blocking with mAB, labeled target cells were preincubated for 30 min with saturating amounts of 2C10, 6D4 or W6/32 ascites before exposure to T cells. Anti-TCR Vδ1 mAB δTCS1 or control IgG1 was added at 10 μg/ml to T cells 30 min before addition of labeled targets. The assays were done at constant effector to target ratios of 5 to 1.

Monoclonal Antibody Production

mAB 6D4 was generated by immunization of RBF/DnJ mice (Jackson Laboratories) with mouse LTK-MICA transfectants as described (Groh et al., 1996), and identified by screenings of hybridoma supernatants by indirect immunofluoresence stainings and flow cytometry of C1R, C1R-MICA and C1R-MICB transfectants. 6D4 was subcloned twice and is of the IgG1 isotype.

Heat Shock Treatment

For heat shock treatment, the adherent cells grown in Petri dishes were floated in a 42° C. waterbath for 90 min. One hour later, total cellular RNAs were prepared, gel-fractionated and blot hybridized with [³²P]dCTP-labeled DNA probes for MICA, HLA-B and hsp70 as described (Groh et al., 1996). For surface staining with mAB and cytotoxicity assays, cells were harvested 16 h after heat shock induction using non-enzymatic cell dissociation solution (Sigma, St. Louis, Mo.).

Metabolic Labeling

C1R-MICA cells (2×10⁷) were labeled with a mixture of 15 [³H]-labeled amino acids (Code TRK440; Amersham) and proteins solubilized in 2 ml NP40 lysis buffer in the presence of protease inhibitors. After preclearing with Protein A-Sepharose (Pharmacia), MICA and MHC class I (C1R cells express only HLA-C) were each precipitated from one half of the lysate using ascites of mAB 2C10 and W6/32 (Parham et al., 1979), respectively. Washed immunoprecipitates were incubated in 0.2 ml 1M acetic acid for 15 min and fractionated by gel filtration using a Biogel P10 column (Biorad; equilibrated with 1M acetic acid, 1%-bovine-serum albumine and 0.1% NP40). 0.5 ml fractions were collected and radioactivity counted.

Results and Discussion

Vδ1 γδ T cells isolated by cell sorting were grown as two lines, δ1A and δ1B, which were cultured in the presence of cytokines and irradiated C1R cells transfected with complementary DNA (cDNA) for MICA or MICB, respectively.

After 4 weeks of repeated stimulation and expansion, the T cell lines were tested for phenotype and function. They were homogeneously positive for Vδ1 γδ TCR and negative for CD4 and CD8. As is characteristic of intestinal intraepithelial T cells, they expressed the α^{E}β₇ integrin (Cerf-Bansussan et al., 1987; Cepek et al., 1994).

In chromium release assays with labeled C1R-MICA or C1R-MICB cells as targets, the δ1A and δ1B T cells were cytotoxic against both of these transfectants but not against untransfected C1R cells. Two CD8⁺αβ T cell lines grown and tested under identical conditions gave negative results. The same observations were made with T cell lines from a second intestinal epithelial tumor.

The inventors further analyzed the apparent recognition of MICA and MICB by the intestinal epithelial Vδ1 γδ T cells using the δ1B line. MICA transfectants of Daudi cells, which lack β₂m and surface MHC class I (Arce-Gomez, 1978), were as effectively lysed as Daudi-β₂m-MICA double transfectants with normal expression of class I. Moreover, transfectants of the lymphoblastoid cell line mutant 5.2.4, which lacks expression of most MHC class II molecules (Mellins et al., 1991), and of mouse T cell lymphoma EL4 cells were recognized. No lytic activity was observed against B cell lines with diverse MHC haplotypes. Thus, the δ1B T cell responses were independent of the presence of MHC class I and class II and were not secondary to crossreactivity with some alleles of these molecules.

Cytotoxicity against the transfected target cells was inhibited when these were preincubated with monoclonal antibodies (mAB) 2C10 or 6D4, which are specific for MICA, and MICA and MICB, respectively (Groh et al., 1996). Treatment with the anti-class I mAB W6/32 or isotype control IgG had no effect (Parham et al., 1979).

The epitopes recognized by mAB 2C10 and 6D4 are within the α1α2 domains of MICA and MICB, as determined by immunofluorescence stainings of C1R transfectants expressing mouse class I H-2D^{b} or K^{b} hybrid molecules in which the α1α2 or α3 domains have been substituted with the corresponding sequences of MICA (Groh et al., 1996). Accordingly, the δ1B T cells lysed C1-MICAα1α2-D^{b} but not C1R-MICAα3-K^{b} cells. Thus, Vδ1 γδ T cells derived from intestinal epithelium were restricted by MICA and MICB and recognized an epitope or epitopes associated with the α1α2 domains of these molecules.

The inventors examined whether the recognition of MICA involved antigen processing and the presentation of peptide ligands. With conventional MHC class I, the peptides are mainly generated by proteasomes in the cytosol and translocated into the endoplasmic reticulum by the trasporters associated with antigen processing (TAP) (York and Rock 1996; Koopmann et al., 1997). Treatment of C1R-MICA cells with lactacystin (target cells were exposed to 10 or 50 μM latacystin (Calbiochem) for 2 h before labeling and subsequent testing with the δ1B T cells (Fenteany et al., 1995;. Cerundolo et al., 1997)), which irreversibly blocks proteasome functions, had no effect on the recognition of MICA by the δ1B T cells. But this did not preclude the presence of long-lived MICA-peptide complexes. However, the transfected mutant 5.2.4 cells, which lack TAP, were also proficient targets, hence arguing against a function of MICA in the MHC class I pathway. Because the cytoplasmic tail sequences of MICA and MICB have no discernible motifs for endosomal targeting or internalization (Sandoval and Bakke, 1994), an acquisition of peptides in endosomal vesicles, similar to MHC class II (Cresswell, 1994), was unlikely.

The inventors sought physical evidence for MICA-peptide complexes by gel filtration chromatography of acid-dissociated immunoprecipitates that were isolated with mAB 2C10 from lysate of C1R-MICA cells after metabolic labeling with tritiated amino acids. The eluted fractions contained a single peak of radiolabeled polypeptide that was of high molecular weight and corresponded to MICA. By contrast, analysis of MHC class I complexes isolated with mAB W6/32 yielded labeled fractions of high and low molecular weights. Thus, under these experimental conditions, there was no evidence for an association of MICA with peptides. This was consistent with its recognition by the δ1B T cells independent of β₂m and with no requirement for conventional class I antigen processing.

These results were in agreement with previous models of antigen recognition by γδ T cells (Holoshitz et al., 1993; Weintraub et al., 1994; Morita et al., 1995; Rock et al., 1994; Schild et al., 1994) and supported a role of MICA and MICB as self antigens. The inventors used intestinal epithelial cell lines to investigate the expression, regulation and T cell recognition of these molecules. Semiconfluent Lovo, HCT116 and DLD-1 cells that were rapidly proliferating expressed large amounts of MICA and MICB, as determined by staining with mAB 2C10 and 6D4 (Cell lines Lovo, HCT116, DLD-1, HUTU-80 and SW480 were from the American Type Culture Collection (ATCC)). As with the transfected target cell lines, they were lysed by the SIB T cells in a specific interaction that was inhibited by mAB 6D4. Other intestinal epithelial cell lines (HUTU-80 and SW480) gave similar results.

Previous evidence has suggested that the expression of MICA in intestinal epithelium may not be constitutive but stress-induced (Groh et al., 1996). In proliferating cell lines (Milarski et al., 1986), however, transcription of the hsp70 gene is activated in the absence of cellular stress. The inventors used Lovo, HCT116 and HUTU-80 cells grown as non-proliferating confluent monolayers to investigate the expression of MICA and MICB before and after heat shock induction. Uninduced cells had very low steady-state levels of MICA and MICB mRNA and expressed small amounts of the encoded surface molecules. But heat shock induction resulted in large increases of mRNA and protein expression.

Concurrently, hsp70 mRNA was potently induced, whereas class I HLA-B mRNA and surface class I HLA-A, -B and -C detected with mAB W6/32 on HCT116 and HUTU-80 cells (Lovo lacks β₂m and thus class I surface expression) were not significantly changed. This induced expression of MICA and MICB was functionally significant since the heat shock treated Lovo, HCT116 and HUTU-80 cells were sensitized to lysis by the δ1B T cells, whereas no or minimal lytic activity was observed with the uninduced cells at high effector to target ratios. As with the proliferation cell lines, cytotoxicity was inhibited by mAB 6D4. Thus, the expression of MICA and MICB and their recognition by the δ1B T cells were regulated by cell stress. Moreover, because these results were obtained with cell lines derived from intestinal epithelium, which is the only peripheral site where expression of MICA has been observed (Groh et al., 1996), MICA and presumably MICB were functionally associated with Vδ1 γδ T cells in this compartment.

The inventors investigated whether MICA and MICB were recognized by T cells expressing a particular γδ heterodimer or diverse TCR and sought evidence for TCR engagement. A total of 16 T cell clones derived from the δ1A and δ1B lines, including 8 clones from each, showed functional activity against C1R-MICA and C1R-MICB targets. Analysis of γ and δ chain cDNA sequences that were obtained by reverse transcription-polymerase chain reaction (RT-PCR™) with specific oligonucleotide primers identified altogether 5 distinct γ and δ chain pairs.

The γ chains included Vγ1.3, 1.4, 1.5 or 1.8, and Jγ2.1 or 2.3. All of the & chains expressed Vδ1 and Jδ1 with diverse junctions encoded by one or two D segments and nontemplated N region nucleotides (Porcelli et al., 1991; Hata et al., 1988; Loh et al., 1988). These data indicated that MICA and MICB were broadly recognized by Vδ1 γδ T cells expressing diverse TCR and supported an engagement of these molecules by these TCR.

In conclusion, these results define a novel T cell-MHC ligand interaction. It has previously been found that intestinal epithelial Vδ1 γδ T cells recognize epithelial cell lines without restriction by polymorphic MHC class I or class II molecules (Maeurer et al., 1996). As shown here, stress-induced MHC class I-related molecules, MICA and MICB, function as target antigens recognized by these T cells. A number of allelic variants of uncertain significance of MICA has been identified (Fodil et al., 1996). However, the inventors observed no differences in the recognition by the δ1 B T cells of C1R transfectants expressing 3 alleles representing most of the sequence variation in the α1α2 domains of MICA. The inventors tested C1R cDNA transfectants expressing MICA alleles 1,4 and 5 (Groh et al., 1996; Fodil et al., 1996).

Although MICA and MICB are encoded in the MHC, their recognition was “MHC-unrestricted”. This was in accord with the recognition of all of the intestinal epithelial cell lines tested. Because MICA and MICB were functionally monomorphic and recognized on diverse target cells without an apparent requirement for antigen processing, and because there was no evidence for associated peptide ligands, it seems probable that these molecules alone conferred specificity in the recognition by the Vδ1γδ T cells. This would be consistent with current models of γδ T cell recognition of antigen (Holoshitz et al., 1993; Weintraub et al., 1994; Morita et al., 1995; Rock et al., 1994; Schild et al., 1994), but remains tentative until the absence of peptide or non-peptide ligands is conclusively demonstrated. The stress-induced expression of MICA and MICB and their broad recognition by diverse Vδ1 γδ T cells may serve an immune surveillance mechanism for the detection of damaged, infected or transformed intestinal epithelial cells, or stimulate T cell secretion of growth factors for the maintenance of epithelial homeostasis (Boismenu and Havran, 1994). The previously noted irregular distribution of MICA in variable areas of intestinal epithelium may reflect such an induction (Groh et a, 1996).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Alexander et al., Immunogenetics, 31:169-178, 1990.

An et al., Proc. Amer. Assn. Canc. Res., 36:82, 1995.

Araki et al., Proc., Natl. Acad Sci. USA, 85:679-683, 1988.

Arce-Gomez et al., Tissue Antigens, 11:96-112, 1978.

Arden et al., Immunogenetics, 42:455, 1995.

Arnold et al., Nature, 360:171, 1992.

Asarnow et al., Cell, 55:837, 1988.

Augustin et al., Nature, 340:239-241, 1989.

Bahram and Spies, Immunogenetics, 43:230-233, 1996.

Bahram et al., Immunogenetics, 44:80-81, 1996.

Bahram et al., Proc. Natl. Acad Sci. USA, 91:6259-6263, 1994.

Baichwal and Sugden, In: Kucherlapati R, ed. Gene transfer. New York: Plenum Press, pp. 117-148,1986.

Balk et al., Science, 265:259-262, 1994.

Barany and Merrifield, The Peptides, Gross and Meienhofer, eds., Academic Press, New York, pp. 1-284 1979.

Barbosa, et al., Proc. Natl. Acad. Sci. USA, 79:6327-6331, 1982.

Beckman et al., Nature, 372:691, 1994.

Bellus, J., Macromol. Sci. Pure Appl. Chem, A31(1):1355-1376, 1994.

Benvenisty and Neshif, Proc. Nat. Acad. Sci. USA, 83:9551-9555, 1986.

Bjorkman and Parham, P. Annu. Rev. Biochem., 59:253-288, 1990.

Boismenu and Havran, Science, 266:1253, 1994.

Borst et al., J. Exp. Med., 167:1625,1988.

Brinster et al., Proc. Nat'l Acad. Sci. USA, 82:4438-4442, 1985.

Bukowski et al., J. Immunol., 154:998, 1995.

Campbell et al., J. Mol. Biol., 180:1-19, 1984.

Capaldi et al., Biochem. Biophys. Res. Comm., 76:425, 1977.

Carter and Flotte, Curr. Top. Microbiol. Immunol., 218:119-144, 1996.

Cepek et al., Nature, 372:190, 1994.

Cerf-Bensussan, et al., Eur. J Immunol., 17:1279, 1987.

Cerundolo, et al., Eur. J Immunol., 27:336, 1997.

Chang et al., Hepatology, 14:134A, 1991.

Chatterjee et al., Ann. N. Y Acad. Sci., 770:79-90, 1995.

Chen and Okayama, Mol. Cell Biol., 7:2745-2752, 1987.

Chen et al., Biotechnology, 13:1191-1197, 1995.

Chien and Davis, Immunol. Today, 14:597, 1993.

Chowers, et al., J. Exp. Med, 180, 183-190, 1994.

Coffin, In: Virology, Fields et al. (eds.), New York: Raven Press, pp. 1437-1500, 1990.

Cooke et al., Cell, 27:487-496, 1981.

Cotton, R., Biochem J., 263:1-10,1989.

Couch et al., Am. Rev. Resp. Dis., 88:394-403, 1963.

Coupar et al., Gene, 68:1-10, 1988.

Craig and Gross, Trends Biochem. Sci., 16:135-139, 1991.

Cresswell, Annu. Rev. Immunol., 12:259, 1994.

Dausset and Svejgaard, (eds) HLA and Disease, pp316, Munksgard, Copenhagen, 1977.

Davis and Bjorkman, Nature, 334:395, 1988.

De Libero et al., J. Exp. Med, 173:1311, 1991.

Deusch et al., Eur. J. Immunol., 21:1053-1059,1991.

Donahue et al., J. Biol. Chem., 269:8604-8609, 1994.

Dubensky et al., Proc. Nat. Acad. Sci. USA, 81:7529-7533, 1984.

Ebert, Gastroenterology, 97:1372, 1989.

Ellis et al., Nucleic Acids Res., 22:2710-2711, 1994.

EPO 320,308

EPO 329,822

Fechheimer et al., Proc. Nat'l Acad. Sci. USA, 84:8463-8467, 1987.

Fenteany et al., Science, 268:726, 1995.

Ferkol et al., FASEB J., 7:1081-1091, 1993.

Ferrari et al., J ViroL, 70:3227-3234, 1996.

Ferrick et al., Cell, 57:483492, 1989.

Ferrick et al., J. Immunol., 145:20-27, 1990.

Fisher et al., J. Virol., 70:520-532, 1996.

Flotte et al., Proc. Natl. Acad. Sci. USA, 90:10613-10617, 1993.

Fodil et al., Immunogenetics, 44:351-357, 1996.

Forster and Symons, Cell, 49:211-220, 1987.

Fraley et al., Proc. Nat'l Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Freshner, “Animal Cell Culture: a Practical Approach”, Second Edition, Oxford/New York, IRL Press, Oxford University Press, 1992.

Friedmann, Science, 244:1275-1281, 1989.

Frobman, PCR PROTOCOLS: A GUIDE TO METHODS AND APPLICATIONS, Academic Press, N.Y., 1990.

GB Application No. 2,202,328

Gefter et al., Somatic Cell Genet., 3:231-236, 1977.

Geraghty, D. E., Curr. Opin. Immunol., 5:3-7, 1993.

Gerlach et al., Nature, 328:802-805, 1987.

Germain and Margulies, Annu. Rev. Immunol., 11:403, 1993.

Ghosh-Choudhury et al., EMBO J., 6:1733-1739, 1987.

Ghosh and Bachhawat, In: Wu G. and C. Wu ed. Liver diseases, targeted diagnosis and therapy using specific receptors and ligands. New York: Marcel Dekker, pp. 87-104, 1991.

Gibbs and Caskey, Science, 236: 303-305, 1987.

Goding, In Monoclonal Antibodies: Principles and Practice, 2d ed., Orlando, Fla., Academic Press, pp. 60-61, and 71-74, 1986.

Gomez-Foix et al., J. Biol. Chem., 267:25129-25134, 1992.

Goodman et al., Blood, 84:1492-1500, 1994.

Gopal, Mol. Cell Biol., 5:1188-1190, 1985.

Graham and Prevec, Biotechnology,-20:363-390, 1992.

Graham and Prevec, In: E. J. Murray (ed.), Methods in Molecular Biology: Gene Transfer and Expression Protocol, Clifton, N.J.: Humana Press, 7:109-128, 1991.

Graham and van der Eb, Virology, 52:456467, 1973.

Graham et al., J. Gen. Virol., 36:59-72, 1977.

Grandea et al., Science, 270:105-108, 1995.

Grandea III, et al., Science 270:105, 1995.

Groh et al., Proc. Natl. Acad. Sci. U.S.A, 93:12445, 1996.

Gross et al., J. Exp. Med., 180:1337-1344, 1994.

Gross et al., Proc. Nat'l. Acad. Sci., 86:4107-4111, 1989.

Grossberger and Parham, Immunogenetics, 36:166-174, 1992.

Grunhaus and Horwitz, Seminar in Virology, 3:237-252, 1992.

Halbert et al., J. Virol., 65: 473478, 1991.

Hammerling, et al., Proc. Natl. Acad. Sci. USA, 79:4737-4741, 1982.

Hanau et al., Hum. Immunol., 41:61-68, 1994.

Happ et al., Nature, 342:696-698, 1989.

Haregewoin et al., Nature, 340:309-312, 1989.

Harland and Weintraub, J. Cell. Biol., 101:1094-1099, 1985.

Hata et al., Science, 240:1541, 1988.

Hedrick et al., Nature, 308:153-158, 1984.

Hermonat and Muzycska, Proc. Nat'l Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Holcombe et al., J. Exp. Med., 181:1433-1443, 1995.

Holoshitz et al., Nature, 339:226-229, 1989.

Holoshitz et al., Int. Immunol., 5:1437, 1993.

Holoshitz et al., Science, 219:56-58, 1983.

Holtmeier et al., J. Immunol., 158:5632, 1997.

Horton, et al, Biotechniques, 8:528-533, 1990.

Horwich et al., J. Virol., 64:642-650, 1990.

Howell and Lane, Antibodies: A Laboratory Manual (Cold Spring Harbor Laboratory, Cold Spring Harbor, N.Y.)., 1988.

Hunt and Morimoto, Proc. Natl. Acad. Sci. USA, 82:6455-6459, 1985.

Innis et al., PCR Protocols, Academic Press, Inc., San Diego Calif., 1990.

Janeway et al., Immunol. Today, 9:73, 1988.

Janis et al., Science, 244:713-716, 1989.

Johnson et al., In BIOTECNOLOGY AND PHARMACY, Pezzuto et al., Eds., Chapman and Hall, New York 1993.

Jones and Shenk, Cell, 13:181-188, 1978.

Joyce, Nature, 338:217-244, 1989.

Kabelitz et al., J. Exp. Med., 171:667, 1990.

Kagnoff, Gastroenterology, 105:1275-1280, 1993.

Kaliyaperumal et al., J. Immunol., 155:2379-2386,1995.

Kaneda et al., Science, 243:375-378, 1989.

Kanei-Ishii et al., Science, 277:246, 1997.

Kaplitt et al., Arm. Thor. Surg., 62:1669-1676, 1996.

Kaplitt et al., Nat. Genet., 8:148-153, 1994.

Karlsson et al., EMBO J., 5:2377-2385, 1986.

Kato et al., J. Biol. Chem., 266:3361-3364, 1991.

Kaufman et al., Immunol. Today, 11:129-136, 1990.

Kaur and McDougall, J. Virol., 62:1917-1924, 1988.

Kaur et al., J. Gen. Virol., 70:1261-1266, 1989.

Kearney, et al., J. Immunol., 123:1548-1550, 1979.

Kessler et al., J. Leukocyte Biol., 60:729-736, 1996.

Kim and Cooke, Proc. Natl. Acad. Sci. USA, 84:8788-8792, 1987.

Klein et al., Nature, 327:70-73, 1987.

Klein, et al., Proc. Natl. Acad. Sci. USA, 91:6251-6252, 1994.

Koeberl et al., Proc. Natl. Acad. Sci. USA, 94:1426-1431, 1997.

Kohler and Milstein, Eur. J. Immunol., 6:511-519, 1976.

Kohler and Milstein, Nature, 256:495-497, 1975.

Koning et. al., Eur. J. Immunol., 19:2099, 1989.

Koopmann et al., Curr. Opin. Immunol., 9:80, 1997.

Kozak, M., J. Biol. Chem., 266:19867-19870, 1991.

Kwoh et al., Proc. Nat. Acad. Sci. USA, 86:1173, 1989.

Kyte and Doolittle, J. Mol. Biol., 157(1):105-132,1982.

Le Gal La Salle et al., Science, 259:988-990, 1993.

Lehner and Creswell, Curr. Opin. Immunol., 8:59-67, 1996.

Levrero et al., Gene, 101:195-202, 1991.

Liang and Pardee, Science, 257: 967-971, 1992.

Liang et al., J.B.C, 266:6999-7007, 1991.

Lishanski et al., Proc. Nat'l. Acad. Sci USA., 91:2674-2678, 1994.

Loh et al., Proc. Natl. Acad. Sci. USA., 85:9714, 1988.

Lundqvist et al., Int. Immunol., 7:1473, 1995.

Macejak and Sarnow, Nature, 353:90-94, 1991.

Maeurer et al., J. Exp. Med, 183:1681, 1996.

Manipulating the Mouse Embryo; A Laboratory Manual” 2nd edition (eds., Hogan, Beddington, Costantimi and Long, Cold Spring Harbor Laboratory Press, 1994.

Mann et al., Cell, 33:153-159, 1983.

Markowitz et al., J. Virol., 62:1120-1124, 1988.

Martin et al., Proc. Natl. Acad. Sci. US. A, 83:9154, 1986.

McCown et al., Brain Res., 713:99-107, 1996.

Mellins et al., J. Exp. Med, 174:1607-1615, 1991.

Melton et al., Nucleic Acids Res., 12:7035-7056, 1984.

Merrifield, Science, 232:341-347, 1986.

Michel and Westhof, J. Mol. Biol., 216:585-610, 1990.

Milarski and Morimoto, Proc. Natl. Acad. Sci. USA., 83:9517, 1986.

Mizukami et al., Virology, 217:124-130, 1996.

Modlin et al., Nature, 338:544-546, 1989.

Mok et al., Gyn. Oncology, 52:247-252, 1994.

Morimoto, Science, 259:1409-1410, 1993.

Morita et al., Immunity, 3:495, 1995.

Mulligan, Science, 260:926-932, 1993.

Myers and Maniatis, Cold Spring Harbor Symposium on Quantitative Biology, Vol. II, pp. 18275-18284, 1986.

Myers and Maniatis, Science, 230:1242-1246, 1985.

Myers, EPO 0273085

Nicolas and Rubinstein, In: Vectors: A survey of molecular cloning vectors and their uses, Rodriguez and Denhardt (eds.), Stoneham: Butterworth, pp. 494-513, 1988.

Nicolau and Sene, Biochem. Biophys. Acta, 721:185-190, 1982.

Nicolau et al., Methods Enymol., 149:157-176, 1987.

O'Brien et al., Cell, 57:667-667, 1989.

Ohara et al., Proc. Nat'l Acad. Sci. USA, 86:5673-5677, 1989.

Ozato and Sachs, J. Immunol, 125:2473-2477, 1980.

Parham et al., Immunogenetics, 18:1-16,1983.

Parham et al., J. Immunol, 123:342-349, 1979.

Parham et al., Nature, 279:639-641, 1979.

Paskind et al., Virology, 67:242-248, 1975.

PCT/UC87/00880

PCT/US89/01025

Pelletier and Sonenberg, Nature, 334:320-325, 1988.

Perales et al., Proc. Natl. Acad. Sci. USA, 91:40864090, 1994.

Pignon et al, Hum. Mutat. 3:126-132, 1994.

Ping et al., Microcirculation, 3:225-228, 1996.

Porcelli et al., Nature, 360:593-597, 1992.

Porcelli, et al., Immunol. Rev., 120:137, 1991.

Potter et al., Proc. Nat'l Acad. Sci. USA, 81:7161-7165, 1984.

Racher et al., Biotechnology Techniques, 9:169-174, 1995.

Ragot et al., Nature, 361:647-650, 1993.

Rajaseker et al., Proc. Nat'l Acad. Sci., 87:1767-1771, 1990.

Reinhold-Hurek and Shub, Nature, 357: 173-176, 1992.

Remington's Pharmaceutical Sciences, 15th ed., pp. 1035-1038 and 1570-1580,

Renan, Radiother. Oncol, 19:197-218, 1990.

Rich et al., Hum. Gene Ther., 4:461-476, 1993.

Ridgeway, In: Rodriguez R L, Denhardt D T, ed. Vectors: A survey of molecular cloning vectors and their uses. Stoneham: Butterworth, pp. 467-492, 1988.

Rippe et al., Mol. Cell Biol., 10:689-695, 1990.

Rock et al., J. Exp. Med., 179:323, 1994.

Rosenfeld et al., Cell, 68:143-155, 1992.

Rosenfeld et al., Science, 252:431-434, 1991.

Roux et al., Proc. Natl. Acad. Sci. USA, 86:9079-9083, 1989.

Sadasivan et al., Immunity, 5:103, 1996.

Sager et al., FASEB J., 7:964-970, 1993.

Saito et al., Nature, 312:36-40, 1984.

Sambrook et al., Molecular Cloning: A Laboratory Manual, 2d Ed., Cold Spring Harbor Laboratory Press, Cold Spring Harbor, N.Y., 1989.

Samulski et al., J. Virol., 61(10):3096-3101, 1987.

Sandoval and Bakke, Trends Cell Biol., 4:292, 1994.

Sargent et al., Proc. Natl. Acad. Sci. USA, 86:1968-1972, 1989.

Sarver et al, Science, 247:1222-1225, 1990.

Scanlon et al., Proc Natl Acad Sci USA, 88:10591-10595, 1991.

Schild et al., Cells, 76:29-37, 1994.

Schmidt et al., Eur. J. Cell Biol., 65:229-245, 1994.

Shinnick et al., Infect. Immun. 56:446-451, 1988.

Sieling et al., Science, 269:227, 1995.

Sim et al., Nature, 312:771-773, 1984.

Simister and Mostov, Nature, 337:184-187, 1989.

Soderstrom et al., J. Immunol., 152:6017, 1994.

Spencer et al., Eur. J. Immunol., 1335; 1989.

Spies et al., Nature, 348:744-747, 1990.

Spies et al., Science, 243:214-217, 1989.

Stewart and Young, Solid Phase Peptide Synthesis, 2d. ed., Pierce Chemical Co. 1984.

Stratford-Perricaudet and Perricaudet, In: Human Gene Transfer, Eds, O. Cohen-Haguenauer and M. Boiron, Editions John Libbey Eurotext, France, 1991.

Stratford-Perricaudet et al., Hum. Gene Ther., 1:241-256, 1990.

Stroynowski and Forman, Curr Opin Immunol., 7:97-102, 1995.

Stroynowski, I., Annu Rev Immunol, 8:501-530, 1990.

Tam et al., J. Am. Chem. Soc., 105:6442, 1983.

Tanaka et al., Nature, 375:155, 1995.

Tax et al., Nature, 304:445, 1983.

Teitell et al., Crit. Rev. Immunol., 14:1-27, 1994.

Temin, In: Gene Transfer, Kucherlapati (ed.), New York: Plenum Press, pp. 149-188, 1986.

Theophilus et al., Nucleic Acids Research, 17:(19):7707-7722, 1989.

Top et al., J. Infect. Dis., 124:155-160,1971.

Toyonaga and Mak, Ann. Rev. Immunol., 5: 585-620, 1987.

Tur-Kaspa et al., Mol. Cell Biol., 6:716-718, 1986.

U.S. Pat. No. 4,196,265

U.S. Pat. No. 4,554,101

U.S. Pat. No. 4,683,195

U.S. Pat. No. 4,683,202

U.S. Pat. No. 4,800,159

U.S. Pat. No. 4,873,191

U.S. Pat. No. 4,946,773

U.S. Pat. No. 5,262,311

U.S. Pat. No. 5,279,721

U.S. Pat. No. 5,354,855

Van Kerckhove, et al., J. Exp. Med, 175:57-63, 1992.

Varmus et al., Cell, 25:23-36, 1981.

Wagner et al., Science, 260:1510-1513, 1990.

Walker et al., Proc. Nat'l Acad. Sci. USA, 89:392-396 1992.

Waneck et al., J. Exp. Med., 165:1358-1370, 1987.

Wang et al., Cell, 66:335-345, 1991.

Wanke et al., JBC, 6068-6072, 1991.

Watanabe et al., J. Clin. Invest., 95:2945, 1995.

Watson et al., Cancer Res., 54:45984602, 1994.

Weintraub, et al., J. Immunol., 153:3051-3058, 1994.

Welsh et al., Nucleic Acids Res., 20:4965-4970, 1992.

Whiteside et al., Int. J. Cancer, 37:803, 1986.

Wickens, M., Trends Biochem. Sci., 15:277-281, 1990.

Winter and Milstein, Nature, 349(6307):293-299, 1991.

Winter and Perucho, Proc. Nat'l Acad. Sci USA, 82:7575-7579, 1985.

WO 88/10315

WO 89/06700

WO 90107641

WO 92/08117

WO 92119771

Wong et al., Gene, 10:87-94, 1980.

Wong et al., Int. J. Oncol., 3:13-17,1993.

Wu et al., J. Immunol., 141:1476, 1988.

Wu and Wu, Adv. Drug Delivery Rev., 12:159-167,1993.

Wu and Wu, Biochemistry, 27:887-892,1988.

Wu and Wu, J. Biol. Chem., 262:4429-4432,1987.

Wu et al., Mol. Cell. Biol., 5:330-341, 1985.

Wu et al., Genomics, 4:560, 1989.

Xiao et al., J. Virol., 70:8098-8108, 1996.

Yanagi et al., Nature, 308: 145-149, 1984.

Yang et al., Proc. Nat'l Acad. Sci. USA, 87:9568-9572,1990.

York and Rock, Annu. Rev. Immunol., 14:369,1996.

Young, Ann. Rev. Immunol., 8:401-420,1990.

Zelenin et al., FEBS Lett., 280:94-96, 1991.

1. A method for detecting a cancer cell in a sample comprising the steps of:
(a) providing said sample; and
(b) identifying MICA or MICB expression in said sample.

2. The method of claim 2, wherein said identifying comprises binding of MICA or MICB by a MICA- or MICB-binding agent.

3. The method of claim 2, wherein said MICA- or MICB-binding agent is a first antibody.

4. The method of claim 2, wherein said first antibody is a bispecific antibody recognizing both MICA and MICB.

5. The method of claim 3, wherein said first antibody is labeled.

6. The method of claim 5, wherein said label is a radiolabel, a fluorescent label, a chemilluminescent label, an enzyme, or a ligand.

7. The method of claim 3, wherein said first antibody is unlabeled and said first antibody is detected by binding of a detection agent to said first antibody.

8. The method of claim 7, wherein said detection agent is a second antibody.

9. The method of claim 8, wherein said second antibody binds to an Fc-region of said first antibody.

10. The method of claim 9, wherein said second antibody is labeled.

11. The method of claim 9, wherein said label is a radiolabel, a fluorescent label, a chemilluminescent label, an enzyme, or a ligand.

12. The method of claim 3, wherein binding of said first antibody is competitive with a second antibody.

13. The method of claim 1, wherein MICA expression is identified.

14. The method of claim 1, wherein MICB expression is identified.

15. The method of claim 1, wherein MICA and MICB expression are identified.

16. The method of claim 1, wherein said identifying comprises amplifying a MICA or MICB transcript.

17. The method of claim 16, wherein said amplifying comprises PCR.

18. The method of claim 17, wherein said amplifying further comprises, prior to said PCR, reverse transcription.

19. The method of claim 17, wherein the PCR product is detected following electrophoretic separation.

20. The method of claim 17, wherein the PCR product is detected following hybridization.

21. The method of claim 17, wherein the PCR is quantitative PCR.

22. The method of claim 1, wherein the sample is selected from the group consisting of lung tissue, skin tissue, muscle tissue, liver tissue, renal tissue, colon tissue, prostate tissue, breast tissue, brain tissue, cervical tissue, pancreatic tissue, stomach tissue, testicular tissue, ovarian tissue or marrow tissue.

23. The method of claim 1, wherein the sample is selected from the group consisting of sputum, blood, semen, plasma, serum, lymphatic fluid, urine and stool.

24. The method of claim 1, wherein the cancer is selected from the group consisting of brain cancer, lymphatic cancer, liver cancer, stomach cancer, testicular cancer, cervical cancer, leukemia, melanoma, head & neck cancer, esophageal cancer, colon cancer, breast cancer, lung cancer, ovarian cancer, prostate cancer and renal cancer.

25. The method of claim 24, wherein the cancer is colon cancer, breast cancer, lung cancer, ovarian cancer, prostate cancer or renal cancer.

26. A method for purifying Vδ1 γδT cells comprising the steps of:
(a) providing a MICA or MICB polypeptide fixed to a support;
(b) contacting said polypeptide with a starting cell population; and
(c) separating said support from said starting cell population to produce a purified Vδ1 γδT cells population.

27. The method of claim 26, further comprising washing said support following step (c).

28. The method of claim 26, wherein said MICA polypeptide is fixed to said support.

29. The method of claim 26, wherein said MICB polypeptide is fixed to said support.

30. The method of claim 26, wherein said MICA and MICB polypeptides are fixed to said support.

31. The method of claim 26, wherein said support is a culture dish.

32. The method of claim 26, wherein said support is a dipstick.

33. The method of claim 26, wherein said support is a test tube.

34. The method of claim 26, wherein said support is a column matrix.

35. The method of claim 26, wherein said support is a bead.

36. The method of claim 26, wherein said support is a filter membrane.

37. The method of claim 26, wherein said starting cell population comprises peripheral blood cells.

38. The method of claim 26, wherein said starting cell population comprises lymph cells.

39. The method of claim 26, wherein said starting cell population comprises purified T-cells.

40. The method of claim 35, wherein said separating comprises centrifugation.

41. The method of claim 26, wherein the purified Vδ1 γδT cell population comprises at least about 75% Vδ1 γδT cells.

42. The method of claim 26, wherein the purified Vδ1γδT cell population comprises at least about 90% Vδ1 γδT cells.

43. The method of claim 26, wherein the purified Vδ1 γδT cell population comprises at least about 95% Vδ1 γδT cells.

44. The method of claim 26, wherein the purified Vδ1 γδT cell population comprises at least about 99.9% Vδ1 γδT cells.

45. The method of claim 26, wherein the purified Vδ1 γδT cell population comprises at least about 99.9% Vδ1γδT cells.

46. A method for enriching a cell population for Vδ1 γδT cells comprising the steps of:
(a) providing a MICA or MICB polypeptide linked to a fluorescent or chemilluminescent label;
(b) contacting said polypeptide with said cell population; and
(c) separating cells exhibiting fluorescence or chemilluminescence.

47. The method of claim 46, wherein said label is selected from the group consisting of fluorescein, rhodamine, green fluorescent protein and luciferase.

48. The method of claim 46, wherein said separating comprises cell sorting.

49. The method of claim 46, wherein said polypeptide is linked directly to said label.

50. The method of claim 46, wherein said polypeptide is linked to said label by a linking moiety.

51. The method of claim 50, wherein said linking moiety is a bead.

52. The method of claim 46, wherein the resulting cell population comprises at least about 99% Vδ1 γδT cells.

53. A method of targeting a therapeutic agent to a tumor cell expressing MICA or MICB on its surface comprising the steps of:
(a) providing a therapeutic agent having a MICA- or MICB-binding agent conjugated thereto; and
(b) contacting said therapeutic agent with said tumor cell.

54. The method of claim 53, wherein said MICA- or MICB-binding agent is an antibody.

55. The method of claim 54, wherein said antibody is humanized murine monoclonal antibody.

56. The method of claim 53, wherein said therapeutic agent is selected from the group consisting of a toxin, a cytokine, a nucleic acid encoding an antitumor agent, a chemotherapeutic and a radionuclide.

57. The method of claim 53, wherein said binding agent binds to MICA.

58. The method of claim 53, wherein said binding agent binds to MICB.

59. The method of claim 53, wherein said binding agent binds to both MICA and MICB.

60. The method of claim 59, wherein said binding agent is a bispecific antibody.

61. A method for treating cancer comprising the step of administering to a subject a therapeutic agent having a MICA- or MICB-binding agent conjugated thereto.

62. The method of claim 61, wherein said MICA- or MICB-binding agent is an antibody.

63. The method of claim 62, wherein said antibody is humanized murine monoclonal antibody.

64. The method of claim 61, wherein said therapeutic agent is selected from the group consisting of a toxin, a cytokine, a nucleic acid encoding an antitumor agent, a chemotherapeutic and a radionuclide.

65. The method of claim 61, wherein said binding agent binds to MICA.

66. The method of claim 61, wherein said binding agent binds to MICB.

67. The method of claim 61, wherein said binding agent binds to both MICA and MICB.

68. The method of claim 67, wherein said binding agent is a bispecific antibody.

69. The method of claim 61, wherein said cancer is selected from the group consisting of brain cancer, lymphatic cancer, liver cancer, stomach cancer, testicular cancer, cervical cancer, leukemia, melanoma, head & neck cancer, esophageal cancer, colon cancer, breast cancer, lung cancer, ovarian cancer, prostate cancer and renal cancer.

70. The method of claim 61, wherein said administering comprises injection.

71. The method of claim 70, wherein said injection is intratumoral.

72. The method of claim 71, wherein said injection is intravenous.

74. A method for expanding Vδ1 γδT cells in a T-cell population comprising the steps of:
(a) contacting said T-cell population with MICA or MICB; and
(b) incubating said T-cell population under conditions permitting the growth and division of T-cells.

75. The method of claim 74, wherein said contacting comprises culturing said T cell population is cultured with cells expressing MICA and MICB.

76. The method of claim 74, wherein said MICA or MICB are purified protiens.

77. The method of claim 74, wherein said starting cell population is selected from the group consisting of peripheral blood cells, lymph cells and purified T-cells.

78. A method of adoptive immunotherapy comprising the step of administering to a patient a population of purified Vδ1 γδT cells.

79. The method of claim 78, wherein said patient suffers from cancer.

80. A method of increasing expression of MICA in a cell comprising providing to said cell an expression construct comprising a coding region for MICA, wherein said coding region is under the control of a promoter active in eukaryotic cells.

81. The method of claim 80, wherein said expression construct is a viral expression vector.

82. The method of claim 80, wherein said expression construct further comprises a polyadenylation signal.

83. The method of claim 80, wherein said cell is a human cell.

84. The method of claim 83, wherein said human cell is in a human subject.

85. The method of claim 84, wherein said human cell is a tumor cell.

86. The method of claim 84, further comprising administering to said human subject a therapeutic agent having a MICA- or MICB-binding agent attached thereto.

87. The method of claim 86, wherein said binding agent is an antibody.

88. A method of increasing expression of MICB in a cell comprising providing to said cell an expression construct comprising a coding region for MICB, wherein said coding region is under the control of a promoter active in eukaryotic cells.

89. The method of claim 88, wherein said expression construct is a viral expression vector.

90. The method of claim 88, wherein said expression construct further comprises a polyadenylation signal.

91. The method of claim 88, wherein said cell is a human cell.

92. The method of claim 91, wherein said human cell is in a human subject.

93. The method of claim 92, wherein said human cell is a tumor cell.

94. A transgenic non-human mammal expressing a human MICA polypeptide.

95. The transgenic mammal of claim 94, wherein said mammal is a mouse.

96. The transgenic mammal of claim 95, wherein said mouse expresses human MICA in a tissue specific fashion.

97. The transgenic mammal of claim 95, wherein said mouse selectively expresses MICA in intestinal epithelium.

98. A transgenic non-human mammal expressing a human MICB polypeptide.

99. The transgenic mammal of claim 98, wherein said mammal is a mouse.

100. The transgenic mammal of claim 99, wherein said mouse expresses human MICB in a tissue specific fashion.

102. The transgenic mammal of claim 100, wherein said mouse selectively expresses MICB in intestinal epithelium.

103. A transgenic non-human mammal expressing human MICA and MICB polypeptides.


SEQUENCE LISTING (16 sequences)

SEQ ID NO: 1 - DNA, 11722 bp, Homo Sapiens
cactgcttgagccgctgagagggtggcgacgtcggggccatggggctgggcccggtcttcctgcttctggctggcatcttcccttttgcacctccgggagctgctgctggtgagtggcgttcctggcggtcctcggcggagcgggagcagtgggacgtttccgggggtcgggtgggtagcggcgagcgctgtgcggtcagggcggggctcctgtgccctgtcggtggcgcagggagctggacgcggcccgttaccgccacacttcagccctgcttccccgtcacttttcagtcctcctcgggatcgcgcatcacctgcactttctggtctcctcctgctctttctctcctcgcgtctcctccgcttcctctcacttttcggacaaaccagtccttctgaggcccatgggttcccgggctgcctccggggctgctcctgtgaatggcattcgagtgcccttccagcgcggccactgaagcagccacaacccccggtgctcggggcggctctcaggtccctgaagtcctgtcctctcccggagccgacgtgttctcagctcctgggccgcagctcctggagtaggggccctcctttctcgggacccggagctggtgcttcctgctgctgtggggactgtggggggtcctgactctcaagctgaggggttggagtctgcaggctccgggcagaggattcttcctgcgacttctctcatccccagctcattctcccctcgcctctggctccgagggtcctctcctctctctcatcccacccctactaatgaccagtgatctaaggacaccagattccctctcacctcctccctgcccatctcagggcccgctgagtccttttgccctcccagctccctgctaccccttcctgtgtgctgttctctgatccatttctagggtgtcctctgccctcatcccctgtccccgccaccgaagtccctcctgcaccccttatgggcctttcctacaagcagccttcacccagtgctgcccctatgcctccccgttcccaaatgtccctgactctaactttctggtgctgccttttatccgggggggtcttccctccatcccactcccctccagacccccaaggggaaccctgatgctaatggcagttgggccttaggcagggcgcagggcagcgcagatgccccctcccctccagtgcagatgcctgttctggaccctgcctcattgtggccccttccccactccttcatcctcagcctcaccctcttgaggaccccaccctccagcccacaggtgctggaccatccctccctggtccctccgcccctctccaccttgggaccttgtgctgctcctatctcttgcccagctgccttgggccctcagcacgttctcatctttcagtgggaaagtgggagtgctggagcatatgacagtgctgagcatctttcccaagccccaccctcccccagagcaccctcccctcctgtcctcaccctaccccaagttctcccacagtcactcctgccccatgctcatgccgccctccagttcttgctctgcccatctcccctccccaacccagacctaaaacaggctgttgggccaactgttccttgaccttccttcttttcttttggttccttgaccccagtgggctctcactccccacaccgcatatctaaaatctgttttgcctgctcttggggtgccactgctccccctccagcattactccttttggcaggtccttcctcaggctgagaatctccccctctaccttggttttctctctctggccagcacccccactccttgctttgtttttaatttttaacttttgtttgggtacgtagtagatatatatgtatatatttatggggtacatgggatattttgacacaggcctacaatatgtaataatcacatcagggtaaatgggttatatcacaacaagcatttatcctttctttgtgctacaaacaatcccattatgctctttcagttatttttaaatgtacaataaattattgttgactgtactcaccctgctgtgctatctactagatcttattcattctaattatatttttgtacccattattaaccatccctgctcccccactccccactacccttctcagcctctggtaatcatcattctattgtctctccccatgaggtccattgttttaaattttggctgccacaaataagtgagaacatgcaaagtttgtctgtctgggcctggggcttatttcacttcacaggatgacctccagttctttgcaaatgacacgatggctgaatagttctccacatacacatgtacaccacattttctttatccatgcgtctgttgatggacacttagattgcttgcagatcttggctactttgaatagtgctgcaataaacatggaaaagtagatagctctttaatataccgatttcctttctttggagtatatgcctaacagtgggagtgctggagcatatgacagctctattgtatttttagtttttggaagaacctccacattgtttcccatagtggttgtactagtttacgttcccaccaacagtgtacatcctcaccagcattccttatttctacatcctcgccagcattccttattgcctgtcttctggataaaagccagtttatctggggtgggatgttatctcgtaggagttttgatttgccttcatctgttgacgaatgatgttgagcaccttttcatatacctgtttgccatttatatgtcttcttttgagaaatgactattcagatcttttctcatttttaaattggattattatattttttttcctatagttgttcgagctccttatatgtttcagttactgatcctttgtcagatgaatagtttgaaaatattttctcccattcttggatggtctcttcattttgtttattgtttcctttgctgtgcagaagcctttttacttgatatgatcccatttatgcaattttactttggttacctgtgcttgtggggtattactttaaaaatctttgcccagtccaatatcctagagagtttccccaatgttttcttgtatagtttcatagtttgaggtcatagatttacatctttaatccactttgatttgatttttgtatatggtgaaagacagggtctagtttcattcttctgcataaggatatctagtttccccagcaccatttttgaagagactctcctttgccaatgtgtgttcttggtacctttgttggaaatgagtttactgtagatgtatggaattgtttctgggttctctattctgtttcattggtctgtgtgtctgtttttatgccagtatcatgctgttttggttactgtagctctgtagtataatttgaagtcagataatgtgattcctctagttttgttcattttgctcaggatagctttatctattctggtttttttgtggttccatatgcattttaggattatttttattatttctgtgaagaatgtcattagtgttttgatagggattgcattgaatctgtagattactttgggtagtatggatatttcaacaaaactgattcttccaatccatgaacgtggactatcttttccattttttgtgtccttcaattttttgcatcagtgttttttgtttttggtttttgagatggagtttcactcttgttgcccaggctagaatgcaagggtgtgatcttggctcaccgcaacctccgcctcccaggttcaagctattcttctgcctcagcctcccaagtagctgggattacaggcatgtgccactgtgcctggctaattttctatttttattagagatggggtttctctatgttggccaggctagtcttgaactcctgacctcaggtgatccacctgcctcggcctcccaaagtgctgggattacaggcatgagccaccacgcccagccacatcactgttttatagtttttattggagaggtctttcacttcttcagttaggtttattcctcagtattttattttatttgtagctattgtaaatgggattcgtttcttgatttctttttcagattatttgctgttagcactgatttttgcatgttgattttgtatcctgcaactttactgaatttgttcttcagttctaatggttttttggtggagtctttaggtttttccaaatatcagaccacatgatctgcaaacaaggataatttgacttcttcttttccagttttaatgccctttctttctttctcctgtctgattgctctagttaggatctgcagtactgtgttgcataactgtggtaaaattagtcatccttgtcttattccagatcttagagaaaaggctttcagttttcccccattcagtatgttactagctgtgagtttgtcatatatggcttttattatattgaggtctgttccttgtatacttagttttttgagagtttttatcatgaagggatgttgaatttatcaaatgctttttcagtatcaattgaatgatactggcttttgtcctttattctgttgatatgacgtattacattgattgatttgtgtatgttaaatcatccttgcatacctggaatacattccacttgctcataaagaatgatcttttttaatgtattgttgaatgtggtttgctagtatttccttgacgatttttgcatcggtgttcatcagggatataggcctgtagttttcttttttatgatgtgtctttgcctggtttttgtatcaggatattcctggctttgtaaaatgagtttggaagtattccctcctcctctatttttcagaacagtttgaataggactgacatatgttgttctttaaaagtttaattgtggtaaattatacattacataaattttactgttttaaccacttttaagtgtatactcggtggcattagatacattcacatttttgtgcaacccaaaactctgtgcccattaatcggtaactccccattcctccctacctctggcccctggtaaccaccattctactttttgtttctatgaatttgaccactctaggtacctcatttaagcagaatcatgtaatgtttgtctttttgtttctggcttatttcacttataatatttttgaggttcggtgggcacagtggctcacgcctggatttccagcactttgggaggctgaagcaggtggatcacctgagtttcggagttcgaaaccagcctggccaacatggtgaaaccccatctctactaaaaataataaaagttagccgggcgtgatggcgggtgcctgtaatcccaactacttgggaggctgaggcaggagaatcgcttgaatccgggaagtggaggttgcagtgagctgagatcaggccactgcactccagcctgggcaacaagagtgaaattccatctccaaaaaaaaaaaataaaacaataataataataatatttttgaggttcatccaagttgtagtatgggtcagaatttcattccttttaaggatggataatactcattatatgtatgtaccacatcttggttatccatccctcagacaatggacacttgggttacttctaccttttggatattggcaaatatttcatttcctttgggtatatatttatttcctttgggtatttcttttgggtatatatccagaaatagaagcagtacacaggggcttcattttctctgtctctttgccaaccttgctctgtgtgtgtgtgtatgtgtgtgtgtaggtgtgtgataacagccatcctgattggtttcaggtggcatctcattgtggtttggatttgcattttcctaatgagtgctgatattgagcatcttttcatgtgtttgttgatcatttgtaattttctttgaagaattggccatttaagtcttttgcccattttttcccccacatagcttctcttatcagatatatgacttgcaatatttatttcatttcggggttgattgctttttcactctgattgtgccctttgatgcatagatgttttgaattttcatcagtctactttgtcagttctttctattctatctgtgctttggtgtcatatccatgaaagcactgtcaaatcctatgtcatgaacattatccccaatgtttgcttctaagaaatttttaggttttagttcttgagtgtagagtttaggtctttgattcattttgagttaatttttgtatatagtgcaaattaagggtccaattttattttaacaccccctgcccccagaactatttgctgaaaagatcaactgactctttgtcacctgctcaccccagtggacactagctgttccatccaattgctgtcctggggccttgtcatgctactcttccactttgaacccaagcccacaccgttcgttgctcccctctgggatactgaccccactataaacttctctggggctacaaccttcctaccctttgtgcctcatgaccaccccctcccttgtccccgccatgcccatgatgagtctcttctcgaggcagctccccttgcctccatctcaccctcagcctatgcaccacagccacactggacatgggtccctctgagcctgagtcccttcccattcccaccatctcctctggcaagaccttccttccaccaccttcatgctcctcccttgcccctgcagggcagcctctccccttggcccctattcccttagggggcttgtggccacccagtccttgcacctggcctacaagtttgccatcttcattcccccttcttctgttcatcagccccctcctctatcctcccaccctcacagttttctttgtatatgaaatcctcgttcttgtccctttgcccgtgtgcatttcctgccccaggaaggttgggacagcagacctgtgtgttaaacatcaatgtgaagttacttccaggaagaagtttcacctgtgatttcctcttccccagagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaaggtgagagtcggcaggggcaagagtgactggagaggccttttccagaaaagttaggggcagagagcagggacctgtctcttcccactggatctggctcaggctgggggtgaggaatgggggtcagtggaactcagcagggaggtgagccggcactcagcccacacagggaggcatgggggagggccagggaggcgtaccccctgggctgagttcctcacttgggtggaaaggtgatgggttcgggaatggagaagtcactgctgggtgggggcaggcttgcattccctccaggagattagggtctgtgagatccatgaagacaacagcaccaggagctcccagcatttctactacgatggggagctcttcctctcccaaaacgtggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccagcgtagtcctgaggagaagaggtacggacgctggccaggggctctcctctccctccaattctgctagagttgcctcacctccaagatgtgtccagggaaaccctccctgtgctatggatgaaggcatttcctgttggcacatcgtgtcctgattttcctctattgttagagccactggataaagacagtgggtcagggactggaccatccagtgttgtaatcagggcaagtagaggaccctccgacagaatcctgagcctgtggtgggtgtcaggcaggagaggaagccttcagggccagggctgccccctctgcctcccagcctgcccatcctggagagttccctcctggccccacaacccaggagtccacccctgacatccccctcctcagcatcaatgtggggatcccagagcctgaggccacagtcccaaggcccatcctcctgccagcctggaagaactgggccccagagtgaggacagacttgcaggtcaggggtcccggagggcttcagccagagtgagaacagtgaagagaaacagccctgttcctctcccctccttagaggggagcagggcttcactggctctgccctttcttctccagtgccccccatggtgaatgtcacccgcagcgaggcctcagagggcaacatcaccgtgacatgcagggcttccagcttctatccccggaatatcacactgacctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctggtgagcctagggtgaccctggagagggtcaggccagggtagggacagcagggatggctgtggctctctgcccagtgtataacaagtccctttttttcagggaaagtgctggtgcttcagagtcattggcagacattccatgtttctgctgttgctgctgctgctgctgctatttttgttattattattttctatgtccgttgttgtaagaagaaaacatcagctgcagagggtccaggtgagaaaagcgggcagtttctggagatggtaaggcccctgtctgggcagtagggtcccctcattgctcctgcaaagataggcatgttggtgacaaggcttctgtaacaggggatgaaagttggggaatttgggaagggaatgggggcagcatctccatctacacccataagtgctgcccaagcgagggtcaaacgcccagctgtggcatcttcctgctgcaggtgaggagtgggcagcagggagggctgcggcgcctgctctgtccccatcccggtctctgtgtctcttggactcactagggcgcatccaggtggggtgagctgggaatcacgtgctgaatgctgagggcctggatgatcacggcctcagagggagcaaatagtaaaggcagctgtgatctggggagggccagaaactggagaggaatctgaggagaggcggtgcccctattcccttcctctctgcatccccctcccctgtttctccagccatcggggcggacaccgagaaaaagacctatgaggcccagcctgggggccctgcctgtgtagccctttggagacccctagtaacagggagggtcctgagcacacatggccatctctgtccactgtgcagctccccatgcacctcctccaggagctttcttggggttgtcgtgtcctctgcaccattcgaggccctactctttccaggttcccacggcctggcctccctgagtttcttgcagatgacatggatgagtagataagcagatgtccctgggccatttgaggagtggggcccagcccctcatcagggcagctgtggtccctgttttcatcctacctccgagtgttttcttctccagtccctgagggacacagtcctcagggcccatgtttttggggatttaatctgtgctctgtggcctcaccttgccttccctgagccaatttccctttctaaaggtggtcactgcctggtaagtttggagtaagggacggtcagaatcatttcccctacagtcaggttgtttgatgggggatgaaaagagacagcaggaagttttgtgtttctgcaaagacagaagcagttcaggcgacagtaagaggctggggtgtccaggagggtgtgtctggcagtagggtcgctggtttctcatccttgaacctaattgcactgtcagtcggcccctcaggcctgagcagatgggaaggtttgtcccctgccctgcagcaagagggccctgtccaggaggcacccacaacagaggcagtgcaggtctgtggtcactcctactctcacctgtggcgtctcccgtagagggattgtcagttctggttccctgtgggcaggaatggtttcctcataggtcactggagttttggccaggaaaagagtatgaagttcatgtggcagtttctcaaaattcctgctttcaatgttgatgtccagtaaagatattcgtaatttcagctctataatcttaataggatttcctctaatattgtgaagcatattatatgaaacaggaacacaaatttctcaaaattcctgcgatgtccaataaagattttcataatttcagctctgcaatcttaataggatttcctaatactgtaaagcatattaaatgaaacaggaactcaaatttggagccccctctccaggaggttctgtgtggagatggtggctgtggcagtggcagttcccaggtgcagagggtgggcagaggcagcctcaggctaaggggtctcccctactccacatggagaaaatcccttgtaggttgcaagggcagtggccgggtggaatccctgctagggacagagcaggaaggcctcgcagcctcaccaagcagcagccctggggtggagctgcgtttccagggttaagcggaccaggcaggagtagcggttactcaagagcaggtcacaggcttgggttgtgagggtcaggagaggccaggcctcctcgagcaaggtgggggtcccagggtcaggtcaggtgcagatcctgtggcagccacgtctttccatgctgggcctgctgggccccccaggcttcctgatggggtccccagttaggagctgcctgctcagggctgggaggggaggagcactgagctgcagatagagggcagagcccacagtgggcagggcctgccctggtgtgtaggtgcctctgcaggagaggagggcctggggactgagagcaagggtcagggcctctctttggggaggcctctcactgtaacaggactggtcaggcctgagaggagggcactgggttccctcttgggtcttgtcctttagtcttggggccctttccctccctgcacgatgagtggtgggcacagggcacgggctgatgttgatggagtgatgggagggaactggcaggggctgggaaaagcaaggagggaggaagaaaaaagtgggggcctcatcttccctcagagaaagggcaaatctggttttggagcaactgaagagagaaaagtccccagggaataaacacaacactgcacccagtggagcatttacccatttccctcttttctccagagctcgtgagcctgcaggtcctggatcaacacccagttgggacgagtgaccacagggatgccacacagctcggatttcagcctctgatgtcagctcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 2 - protein, 385 aa, Homo Sapiens
MGLGPVFLLLAGIFPFAPPGAAAEPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPSGKVLVLQSHWQTFHVSAVAAAAAAIFVIIIFYVRCCKKKTSAAEGPELVSLQVLDQHPVGTSDHRDATQLGFQPLMSALGSTGSTEGA

SEQ ID NO: 3 - DNA, 2380 bp, Homo Sapiens
gggccatggggctgggccgggtcctgctgtttctggccgtcgccttcccttttgcacccccggcagccgccgctgagccccacagtcttcgttacaacctcatggtgctgtcccaggatgaatctgtgcagtcagggtttctcgctgagggacatctggatggtcagcccttcctgcgctatgacaggcagaaacgcagggcaaagccccagggacagtgggcagaagatgtcctgggagctaagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacgatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggtgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcgcctagactctacagccaggcggccaggattcaactccctgcctggatctcaccagcactttccctctgtttcctgacctatgaaacagaaaataacatcacttatttattgttgttggatgctgcaaagtgttagtaggtatgaggtgtttgctgctctgccacgtagagagccagcaaagggatcatgaccaactcaacattccattggaggctatatgatcaaacagcaaattgtttatcatgaatgcaggatgtgggcaaactcacgactgctcctgccaacagaaggtttgctgagggcattcactccatggtgctcattggagttatctactgggtcatctagagcctattgtttgaggaatgcagtcttacaagcctactctggacccagcagctgactccttcttccacccctcttcttgctatctcctataccaataaatacgaagggctgtggaagatcagagcccttgttcacgagaagcaagaagccccctgaccccttgttccaaatatactcttttgtctttctctttattcccacgttcgccctttgttcagtccaatacagggttgtggggcccttaacagtgccatattaattggtatcattatttctgttgtttttgtttttgtttttgtttttgtttttgagacagagtctcactcgtcacccaggctgcagttcactggtgtgatctcagctcactgcaacctctgcctcccaggttcaagcacttctcgtacctcagactcccgatagctgggattacagacaggcaccaccacacccagctaatttttgtattttttgtagagacggggtttcgccaagttgaccagcccagtttcaaactcctgacctcaggtgatctgcctgccttggcatcccaaagtgctgggattacaagaatgagccaccgtgcctggcctattttattatattgtaatatattttattatattagccaccatgcctgtcctattttcttatgttttaatatattttaatatattacatgtgcagtaattagattatcatgggtgaactttatgagtgagtatcttggtgatgactcctcctgaccagcccaggaccagctttcttgtcaccttgaggtcccctcgccccgtcacaccgttatcgattactctgtgtctactattatgtgtgcataatttataccgtaaatgtttactctttaaataaaaaaaaaaaaaaa

SEQ ID NO: 4 - protein, 383 aa, Homo Sapiens [sequence omitted]

SEQ ID NO: 5 - DNA, 25 bp, Artificial Sequence
  Synthetic Primer
actggggaacaaggtttatatgaga

SEQ ID NO: 6 - DNA, 24 bp, Artificial Sequence
  Synthetic Primer
tgtcacccgtcttctacaggaccc

SEQ ID NO: 7 - DNA, 17 bp, Artificial Sequence
  Synthetic Primer
ggggccatggggctggg

SEQ ID NO: 8 - DNA, 17 bp, Artificial Sequence
  Synthetic Primer
atctgagatgtcggtcc

SEQ ID NO: 9 - DNA, 25 bp, Artificial Sequence
  Synthetic Primer
cgttcttgtccctttgcccgtgtgc

SEQ ID NO: 10 - DNA, 25 bp, Artificial Sequence
  Synthetic Primer
aacccttcccttacccccgtcgtag

SEQ ID NO: 11 - DNA, 45 bp, Artificial Sequence
  Synthetic Primer
tatgtaaaacgacggccagtttcacctgtgatttcctcttcccca

SEQ ID NO: 12 - DNA, 45 bp, Artificial Sequence
  Synthetic Primer
ggtcttttcaatccccgtctctcgtccagtatcgacaaaggacat

SEQ ID NO: 13 - DNA, 40 bp, Artificial Sequence
  Synthetic Primer
tatgtaaaacgacggccagtttcgggaatggagaagtcac

SEQ ID NO: 14 - DNA, 40 bp, Artificial Sequence
  Synthetic Primer
cgagaggagagggaggttaaccagtatcgacaaaggacat

SEQ ID NO: 15 - DNA, 40 bp, Artificial Sequence
  Synthetic Primer
tatgtaaaacgacggccagtgttcctctcccctccttaga

SEQ ID NO: 16 - DNA, 41 bp, Artificial Sequence
  Synthetic Primer
aaaaagtccctttcacgaccaccagtatcgacaaaggacat